(19) United States
(12) Patent Application Publication
(10) Pub. No.: US 2017/0054098 A1
(43) Pub. Date: Feb. 23, 2017

(54) ORGANIC ELECTROLUMINESCENT ELEMENT

(71) Applicant: KONICA MINOLTA, INC., Tokyo (JP)

(72) Inventors: Takaaki KUROKI, Hachioji-shi, Tokyo (JP); Akihiko TAKEDA, Sagamihara-shi, Kanagawa (JP); Yasunobu KOBAYASHI, Hachioji-shi, Tokyo (JP)

(21) Appl. No.: 15/306,783

(22) PCT Filed: May 12, 2015
(86) PCT No.: PCT/JP2015/063598

§ 371 Date: Oct. 26, 2016

(30) Foreign Application Priority Data
May 22, 2014 (JP) 2014-106050

(51) Int. Cl. H01L 51/50; H01L 51/56; H01L 51/52

(52) U.S. Cl. CPC H01L 51/5012; H01L 51/5268; H01L 51/524; H01L 51/5203; H01L 51/56; H01J 2237/3321; H01J 2237/18; H01J 37/32449

(57) ABSTRACT

Provided an organic electroluminescent element which is configured to comprise: a substrate; a gas barrier layer that is arranged on the substrate; a smooth layer that is mainly composed of an oxide or nitride of Ti or Zr having an amorphous structure; a first electrode; a second electrode; and an organic function layer that is sandwiched between the first electrode and the second electrode. This organic electroluminescent element is able to achieve a good balance between gas barrier properties and flexibility adequacy.

### TECHNICAL FIELD

The present invention relates to an organic electroluminescent element.

### BACKGROUND ART

In recent years, in the field of electronic devices, demands for long-term reliability, a high degree of freedom of shapes, being capable of a curved surface display, and the like have been added to demands for weight reduction and size increase. Accordingly, there have started to be adopted film base materials composed of a transparent plastic or the like instead of a glass substrate which is heavy, fragile, and in which area enlargement is difficult.

However, there is a problem that a film base material composed of transparent plastics or the like have poorer gas barrier properties than a glass substrate. It is found that the use of a substrate having poor gas barrier properties allows intrusion of water vapor and oxygen, and thus, for example, leads to deterioration of a function in an electronic device.

It is generally known that a film having gas barrier properties (gas barrier layer) is formed on a film base material and is used as a gas barrier film, for that reason. For example, a gas barrier film used for a material for wrapping objects that require gas barrier properties and for liquid crystal displays includes a film obtained by vapor deposition of silicon oxide or a film obtained by vapor deposition of aluminum oxide, on a film substrate.

Furthermore, in the organic electroluminescent (EL) element that is one of the electronic devices, it is also known that a light taking-out configuration of providing a light-scattering layer is efficient in order to enhance the light emission efficiency (for example, Patent Literature 1).

However, when the gas barrier layer and a scattering layer are formed on a film substrate, unevenness is generated on the surface. As the result, when an organic function layer including a light-emitting layer is formed thereon, there is caused a problem that degradation of storability under a high temperature and humidity atmosphere, and short circuit (electrical short circuit) are easily generated.

Generally, it is known that the organic EL element is very sensitive to a slight amount of water, oxygen, and other organic substances (remaining solvent, and the like), and there is also proposed a configuration of having a gas barrier layer just beneath the organic function layer (for example, Patent Literature 2).

### CITATION LIST

### Patent Literature

PTL 1: Japanese Patent Laid-Open No. 2004-296437

PTL 2: Japanese Patent No. 4186688

### SUMMARY OF INVENTION

### Technical Problem

As described above, in the organic EL element provided with the gas barrier layer and the light-scattering layer, there is not still any specific/practical disclosure as to achieving a good balance between the gas barrier properties and high efficiency, and curvature-repeating resistance against bending, namely, flexibility adequacy. Therefore, an organic EL element having a high efficiency and achieving a good balance between flexibility adequacy and gas barrier properties has not been realized.

In order to solve the above problem, the present invention provides an organic electroluminescent element which can achieve a good balance between gas barrier properties and flexibility adequacy.

### Solution to Problem

The organic electroluminescent element of the present invention includes: a substrate; a gas barrier layer that is arranged on the substrate; a smooth layer that is mainly composed of an oxide or nitride of Ti or Zr having an amorphous structure; a first electrode; a second electrode; and an organic function layer that is sandwiched between the first electrode and the second electrode.

According to the organic electroluminescent element of the present invention, the smooth layer is composed of the oxide or nitride of Ti having an amorphous structure, or the oxide or nitride of Zr having an amorphous structure, as a main component. It is possible not only to impart flexibility adequacy to the organic electroluminescent element but also to contribute to the enhancement of the gas barrier properties, by provision of such a smooth layer. Accordingly, it becomes possible that the organic electroluminescent element achieves a good balance between gas barrier properties and flexibility adequacy, by provision of the above smooth layer.

### Advantageous Effects of Invention

The present invention provides an organic electroluminescent element which can achieve a good balance between gas barrier properties and flexibility adequacy.

### BRIEF DESCRIPTION OF DRAWINGS

FIG. 1 is a diagram showing a configuration of an organic EL element of the embodiment.

FIG. 2 is a graph showing each element profile of a first gas barrier layer in the thickness direction by an XPS depth profile (distribution in the depth direction) of a layer deposited when a gas inlet moves by 5% in the direction of the film-deposition roller.

FIG. 3 is a diagram showing a configuration of a manufacturing apparatus for forming the first gas barrier layer.

FIG. 4 is a diagram for explaining the movement of the position of the gas inlet in the manufacturing apparatus shown in FIG. 3.

FIG. 5 is a graph showing each element profile in the thickness direction by the XPS depth profile of a layer deposited when the gas inlet moves by 10% in the direction of the film-deposition roller.

FIG. 6 is a graph showing each element profile by the XPS depth profile of a gas barrier layer deposited when the gas inlet is fixed on the perpendicular bisector m of the line segment connecting the film-deposition rollers.

FIG. 7 is a diagram showing a Raman spectroscopic absorption spectrum of TiO₂.

FIG. 8 is a fitting data of a spectroscopic ellipsometer used in the Examples.

FIG. 9 is a graph of wavelength dependence of n (refractive index) and k (extinction coefficient) in the Examples.

### DESCRIPTION OF EMBODIMENTS

Hereinafter, embodiments for carrying out the present invention will be explained, but the present invention is not limited to the following examples.

<Embodiment of Organic Electroluminescent Element>

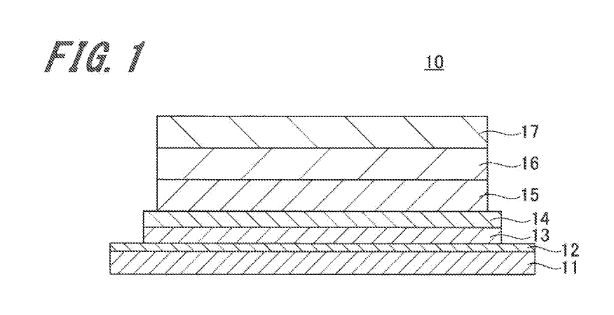

Hereinafter, the specific embodiment of the organic electroluminescent element (organic EL element) of the present invention will be explained. FIG. 1 is a schematic configuration diagram of the organic EL element of the present embodiment.

[Configuration of Organic EL Element]

An organic EL element 10 shown in FIG. 1 is provided on at least one or more gas barrier layer 12 provided on a substrate 11. The organic EL element 10 has a light-scattering layer 13 provided on the gas barrier layer 12 and a smooth layer 14 provided on the light-scattering layer 13. Furthermore, the organic EL element 10 has a first electrode 15 provided on the smooth layer 14, an organic function layer 16 and a second electrode 17.

The organic EL element 10 has a configuration in which the first electrode 15 is made of a transparent electrode and the second electrode 17 acts as a reflective electrode, and is so-referred to as a bottom-emission type configuration in which the light is taken out from the substrate 11 side.

In addition, the organic function layer 16 sandwiched by the first electrode 15 and the second electrode 17 has at least a light-emitting layer containing various organic compounds described below. Additionally, in the light-emitting layer, a positive hole (hole) supplied from one electrode (anode) and an electron supplied from the other electrode (cathode) are recombined to thereby emit light.

The gas barrier layer 12 is provided on the entire surface of the substrate 11 in order to effectively prevent the intrusion of moisture from the substrate 11, and to prolong life of the organic EL element. As shown in FIG. 1, although it is preferable that the gas barrier layer 12 is provided on the side of the substrate 11 on which the element is to be mounted, the gas barrier layers 12 may have a configuration of being provided on both sides of the substrate 11.

There is formed, on the substrate 11 where the gas barrier layer 12 is formed, the light-scattering layer 13 for enhancing the light taking-out efficiency of the organic EL element 10. The light-scattering layer 13 is, for example, composed of a light-scattering particle and a binder. The light-scattering particle preferably has a refractive index higher than that of a material constituting the binder, and, for example, an inorganic particle having a refractive index of 1.6 to 3.0 is preferably used. It is possible to efficiently scatter light and increase the light to be taken out through the substrate 11, by utilizing the difference of the refractive indexes between the inorganic particle having a refractive index of 1.6 to 3.0 and the binder. The refractive index of the scattering layer can be calculated from a volume ratio of each refractive index of the binder and the light-scattering particle.

There is provided, on the light-scattering layer 13, the smooth layer 14 for smoothing the surface of the light-scattering layer 13. As described above, the light-scattering layer 13 has poor surface smoothness due to having the light-scattering particles. Therefore, when the electrodes or the like of the organic EL element 10 is produced on the light-scattering layer 13, various properties of the organic EL element 10 may be lowered. Accordingly, when the light-scattering layer 13 has the configuration of having the light-scattering particles, the smooth layer 14 for smoothing the surface of the light-scattering layer 13 is essential.

In addition, it is important that the smooth layer 14 has a role in guiding light into the light-scattering layer 13 without loss. From this point of view, a refractive index of the smooth layer 14 is preferably close to or the same as that of the organic function layer 16 and the first electrode 15.

Note that it is known that, when each layer from the first electrode 15 or the light-emitting layer up to the smooth layer 14 has a thickness of less than 50 nm, the influence of the refractive index on the layer having a thickness of less than 50 nm is reduced due to an evanescent effect, and thus it is possible to eliminate the refractive index of the layer.

In a case where each layer of the organic EL element is mainly designed by low-molecular organic EL materials having a high refractive index, the thickness thereof is more than 50 nm, and thus it is important that the smooth layer 14 has a high refractive index. Specifically, the refractive index of the smooth layer 14 in the visible light region is preferably 1.6 to 2.5, particularly preferably 1.7 to 2.2, and most preferably 1.75 to 2.

In the organic EL element 10, the smooth layer 14 has an amorphous structure, and is mainly composed of an oxide or nitride of Ti, or an oxide or nitride of Zr.

When the above metal oxide or the metal nitride constituting the smooth layer 14 has the amorphous structure, flexibility adequacy is imparted to the smooth layer 14.

Here, the main component means that a volume ratio of the oxide or nitride of Ti or Zr having the amorphous structure is 50% or more in the smooth layer 14.

Furthermore, the amorphous structure can be detected from spectrum peak defined by a Raman spectroscopic absorption, an X-ray analysis, or the like, and means that a specified Raman peak ratio relative to a crystal structure by thermal crystallization or the like is less than 50%.

In addition, when the smooth layer 14 is formed by using the above metal oxide or the metal nitride as a main component, the effect of effectively preventing the intrusion of moisture is exerted, and thus it becomes possible to prolong the life of the organic EL element 10. Particularly, the smooth layer 14 preferably has a water vapor permeability of less than 0.1 g/(m²·24 h). The smooth layer is formed so as to have a water vapor permeability of more preferably less than 0.05 g/(m²·24 h), particularly preferably less than 0.01 g/(m²·24 h).

In order to further enhance the water vapor permeability of the smooth layer 14 as described above, it is important that the thickness of the smooth layer 14 is increased, and that smoothness of the surface directly under the smooth layer 14 is further enhanced. The thickness of the smooth layer 14 is preferably 20 nm or more, more preferably 50 nm or more, and particularly preferably 100 nm or more. On the other hand, the upper limit of the thickness of the smooth layer 14 is not particularly limited, and is preferably less than 1000 nm, particularly preferably less than 700 nm, from the viewpoint of film absorption. With respect to the smoothness of the surface directly under the smooth layer 14, since the continuous formation of the smooth layer 14 is assumed, a rough surface having a glossiness at 45-45 degrees of less than 10 is not preferable, and the glossiness is preferably 10 or more, more preferably 20 or more, and particularly preferably 30 or more. In addition, in order to avoid the influence of the micro pinholes on the smooth layer 14, an Ra at an AFM of 10 μm×10 μm is less than 30 nm, more preferably less than 20 nm, and particularly preferably less than 10 nm. An Rz is less than 300 nm, more preferably less than 200 nm, and particularly preferably less than 100 nm.

Furthermore, from the viewpoint of long storability, a water vapor permeability Wg of the gas barrier layer 12, a water vapor permeability Ws of the light-scattering layer 13 and a water vapor permeability Wf of the smooth layer 14 preferably satisfy the following conditional equation.

Wg≦Wf<Ws

It is preferable that an absolute value of the water vapor permeability Ws of the light-scattering layer 13 is small. However, since the binder of the light-scattering layer 13 is mainly composed of a resin material, a gas permeability is theoretically large.

On the other hand, it is theoretically possible to enhance the performances of the organic EL element 10 by making the water vapor permeability Wf of the smooth layer 14 smallest. However, since there is a possibility of lowering the light emission efficiency of the organic EL element 10 by the change of the gas absorption amount of the light-scattering layer 13, and there is also a possibility of lowering the film strength.

Therefore, as shown in the above conditional equation, it is preferable that the water vapor permeability Wg of the gas barrier layer 12 is designed so as to be smallest. For example, in order to make the water vapor permeability Wg of the gas barrier layer 12 low, it is also possible to provide a gas barrier layer on a surface of the substrate 11 on an opposite side on which the element is to be mounted. Furthermore, it is also possible to have a configuration of laminating a plurality of basic component members of the gas barrier layer 12.

For example, when the smooth layer 14 is formed by a dry process, a dense layer is easily formed, and thus the gas barrier property is easily enhanced.

In addition, when the smooth layer 14 is formed by a wet process, the smooth layer having a high smoothness is easily formed.

Additionally, from the viewpoint of protecting the light-scattering layer 13 by the minimum number of the smooth layers 14 and of also preventing the intrusion of water vapor or the like in the atmosphere, it is preferable that the light-scattering layer 13 is patterned within the sealing region.

Hereinafter, each configuration of the organic EL element 10 of the present embodiment will be explained. Note that “to” representing the numerical range is used in a sense of including the former and latter numerals as the lower limit and the upper limit.

[Substrate]

The substrate 11 where the organic EL element 10 is provided can include, for example, a resin film, and the like, but is not limited thereto. The substrate 11 to be preferably used can include a transparent resin film.

Examples of the resin films include polyesters such as polyethylene terephthalate (PET) and polyethylene naphthalate (PEN), polyethylene, polypropylene, cellulose esters or derivative thereof such as cellophane, cellulose diacetate, cellulose triacetate (TAC), cellulose acetate butylate, cellulose acetate propionate (CAP), cellulose acetate phthalate and cellulose nitrate, polyvinylidene chloride, polyvinyl alcohol, polyethylene vinyl alcohol, syndiotactic polystyrene, polycarbonate, norbornen resin, polymethylpenten, polyether ketone, polyimide, polyether sulphone (PES), polyphenylene sulfide, polysluphones, polyether imide, polyether ketone imide, polyamide, fluoro resin, Nylon, polymethyl methacrylate, acryl or polyallylates, cycloolefins-based resins such as Alton (commercial name, manufactured by JSR) or APEL (commercial name, manufactured by Mitsui Chemicals), and the like.

[Gas Barrier Layer]

It is preferable that the gas barrier layer 12 is constituted by at least two or more gas barrier layers each having different composition or distribution of constituent elements. According to such a configuration, it is possible to efficiently prevent the permeation of oxygen and water vapor.

The gas barrier layer 12 is preferably a gas barrier film (also referred to as gas barrier membrane, etc.) having a water vapor permeability (25±0.5° C., relative humidity 90±2% RH) measured in accordance with the method of JIS-K-7129-1992 of 0.01 g/(m²·24 h) or less. Furthermore, the film preferably has an oxygen permeability measured in accordance with the method of JIS-K-7126-1987 of 1×10⁻³ ml/(m²·24 h·atm) or less, and a water vapor permeability of 1×10⁻⁵ g/(m²·24 h) or less.

In a case where the gas barrier layer 12 is composed of two or more layers, at least one or more gas barrier layers preferably contains silicon dioxide that is the reaction product of an inorganic silicon compound. Furthermore, at least one or more gas barrier layers of two or more barrier layers preferably contain a reaction product of an organic silicon compound. Namely, at least one gas barrier layer preferably contains, as a constituent element, an element derived from the organic silicon compound, for example, oxygen, silicon, carbon, and the like.

Note that the composition or distribution state of the gas barrier layer 12 of the elements constituting the gas barrier layer 12 may be uniform or different in the direction of layer thickness. When the composition or distribution state of the elements constituting the layer is made different from each other, as described later, it is preferable to make the forming method and the forming material of the gas barrier layer 12 different from each other.

Hereinafter, there will be explained an example where the first gas barrier layer and the second gas barrier layer are formed from the different materials respectively, as the gas barrier layer 12.

(First Gas Barrier Layer)

The constituent elements of the first gas barrier layer may at least contain elements constituting a compound which prevents the permeation of oxygen and water vapor, and may have a different constituent element ratio from the second gas barrier layer described below.

For example, the first gas barrier layer may be provided with a layer which contains, as constituent elements, silicon, oxygen, and carbon on one surface of the substrate 11. In this case, in the distribution curves of the respective constituent elements based on element distribution measurement for the first gas barrier layer in the depth direction by using X-ray photoelectron spectroscopy, it is preferable to be embodied in a form of satisfying all of the requirements (i) to (iv) described below, from the viewpoint of enhancement of gas barrier properties.

(i) The atomic ratio of silicon, the atomic ratio of oxygen, and the atomic ratio of carbon have a magnitude relation indicated below in an area covering 90% or more in the distance area from the surface across the thickness direction of the first gas barrier layer: (atomic ratio of carbon)<(atomic ratio of silicon)<(atomic ratio of oxygen).

(ii) The carbon distribution curve has at least two extreme values.

(iii) The absolute value of the difference between the maximum value and the minimum value of the atomic ratio of carbon in the carbon distribution curve is 5 at % or more.

(iv) In the oxygen distribution curve, the maximum value of the oxygen distribution curve closest to the surface of the first gas barrier layer on the substrate 11 side is the largest value of maximum values of the oxygen distribution curve of the first gas barrier layer.

The first gas barrier layer is preferably a thin film formed on the substrate 11 through plasma enhanced chemical vapor deposition in which, by using a belt-shaped flexible substrate 11, the substrate 11 is conveyed while being in contact with a pair of film-deposition rollers and is subjected to a plasma discharge while a film-deposition gas is supplied between the pair of film-deposition rollers.

Note that the extreme value refers to a maximum value or a minimum value of an atomic ratio of each element to the distance from the surface of the first gas barrier layer in the thickness direction of the first gas barrier layer.

(Definition of Maximum Value and Minimum Value)

The maximum value is a point at which the atomic ratio of an element changes from an increase to a decrease when the distance from the surface of the first gas barrier layer is changed, and at which the value of the atomic ratio of the element decreases by 3 at % or more when the distance from the surface of the first gas barrier layer in the thickness direction of the first gas barrier layer from that point is further changed by 20 nm.

Furthermore, the minimum value is a point at which the atomic ratio of the element changes from a decrease to an increase when the distance from the surface of the first gas barrier layer is changed, and at which the value of the atomic ratio of the element increases by 3 at % or more when the distance from the surface of the first gas barrier layer in the thickness direction of the first gas barrier layer is further changed by 20 nm.

(Relationship Among Average Value, Largest Value, and Smallest Value of Atomic Ratio of Carbon)

The average atomic ratio of carbon in the first gas barrier layer is, as an average value within an entire layer, preferably within the range of 8 to 20 at % from the view point of flexibility, more preferably 10 to 20 at %. When the atomic ratio of carbon is within the above range, it is possible to form the first gas barrier layer sufficiently satisfying both of gas barrier properties and flexibility.

Moreover, the absolute value of the difference between the largest value and the smallest value of the atomic ratio of carbon in the carbon distribution curve of such a first gas barrier layer is preferably 5 at % or more. In addition, in such a first gas barrier layer, the absolute value of the difference between the largest value and the smallest value of the atomic ratio of carbon is more preferably 6 at % or more, particularly preferably 7 at % or more. When the absolute value is 3 at % or more, the barrier properties are sufficient in a case where the first barrier layer is bent.

(Positions of Extreme Values and Relationship Between Largest Value and Smallest Value of Atomic Ratio of Oxygen)

As described above, from the viewpoint of preventing the intrusion of water molecules from the substrate 11 side, in the oxygen distribution curve of the first gas barrier layer, it is preferable that the maximum value of the oxygen distribution curve closest to the surface of the first gas barrier layer on the substrate 11 side takes the largest value of the maximum values of the oxygen distribution curve in the first gas barrier layer.

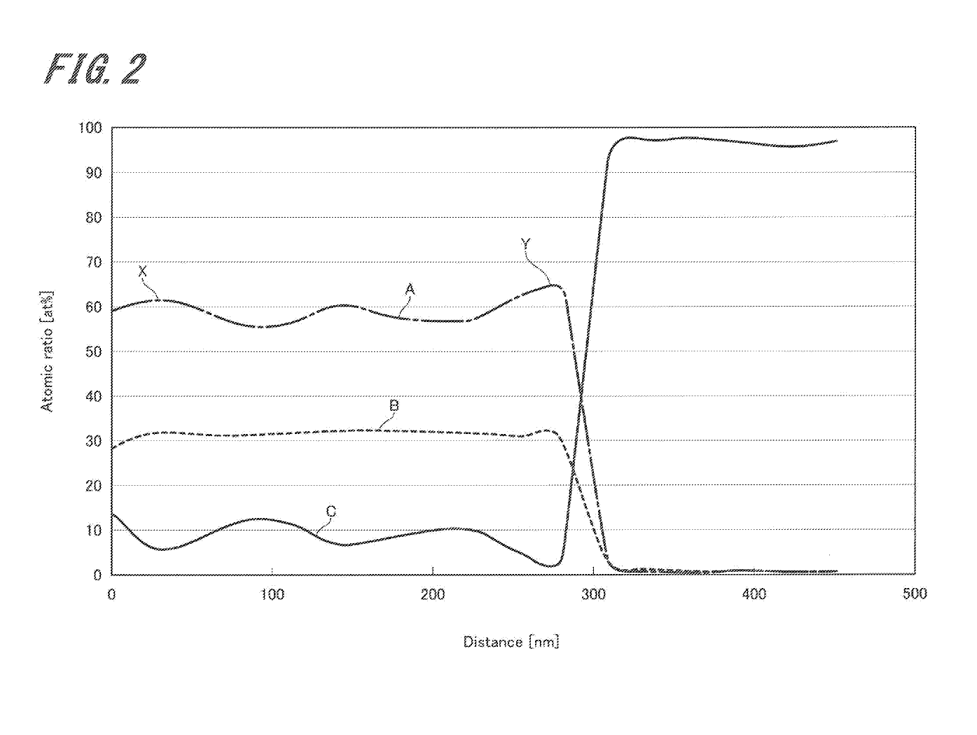

FIG. 2 is a graph illustrating depth profiles of the respective elements in the thickness direction of the first gas barrier layer according to the XPS depth profile (distribution in depth direction).

In FIG. 2, the oxygen distribution curve is designated by A, the silicon distribution curve is designated by B, and the carbon distribution curve is designated by C.

The atomic ratio of each element continuously vary between the surface (distance being 0 nm) of the first gas barrier layer and the surface of the substrate 11 (distance being about 300 nm). However, the fact that the values of the atomic ratio of oxygen is Y>X is preferable from the viewpoint of preventing the intrusion of water molecules from the substrate 11 side, when X is a maximum value of the atomic ratio of oxygen closest to the surface of the first gas barrier layer in the oxygen distribution curve A and Y is a maximum value of the atomic ratio of oxygen closest to the surface of the substrate 11.

The atomic ratio Y of oxygen, being the maximum value in the oxygen distribution curve, closest to the surface of the first gas barrier layer on the substrate 11 side is preferably 1.05 times or more the atomic ratio X of oxygen, being the maximum value in the oxygen distribution curve, closest to the surface of the first gas barrier layer on a side opposite to the substrate 11. Namely, it is preferred that 1.05≦Y/X is established. The upper limit may not be particularly limited, and the upper limit is preferably within the range of 1.05≦Y/X≦1.30, more preferably within the range of 1.05≦Y/X≦1.20. When the upper limit is within this range, the intrusion of water molecules can be prevented, degradation of the gas barrier properties under high temperature and high humidity is not observed, and it is also preferable from the viewpoints of productivity and cost.

Furthermore, in the oxygen distribution curve of the first gas barrier layer, the absolute value of the difference between the largest value and the smallest value of the atomic ratio of oxygen is preferably 5 at % or more, more preferably 6 at % or more, and particularly preferably 7 at % or more.

(Relationship Between Largest Value and Smallest Value of Atomic Ratio of Silicon)

The absolute value of the difference between the largest value and the smallest value of the atomic ratio of silicon in the silicon distribution curve of the first gas barrier layer is preferably less than 5 at %, more preferably less than 4 at %, and particularly preferably less than 3 at %. When the absolute value is within the above range, the obtained first gas barrier layer has sufficient gas barrier properties and the gas barrier layer 12 has sufficient mechanical strength.

(Regarding Composition Analysis in Depth Direction of Gas Barrier Layer by XPS)

The carbon distribution curve, the oxygen distribution curve, and the silicon distribution curve in the direction of thickness (depth) of the first gas barrier layer can be obtained through the so-called XPS depth profile measurement (distribution in the depth direction) in which the surface compositional analysis is sequentially performed while the interior of the specimen is exposed, through the combined use of X-ray photoelectron spectroscopy and ion-beam sputtering using a rare gas such as argon. The distribution curve obtained by such XPS depth profile measurement can be produced by defining a vertical axis as, for example, the atomic ratio (unit: at %) of each element and a horizontal axis as the etching time (sputtering time).

Note that, in the distribution curve of the element in which the horizontal axis is defined as the etching time as described above, the etching time correlates generally with the distance from the surface of the first gas barrier layer in the thickness direction. Therefore, the distance from the surface of the first gas barrier layer can be calculated on the basis of the relationship between the etching rate and etching time adopted in the XPS depth profile measurement, as “the distance from the surface of the gas barrier layer in the thickness direction of the gas barrier layer”.

Furthermore, as the method of sputtering method adopted in the XPS depth profile measurement as described above, it is preferable to employ a rare gas ion sputtering method using argon (Ar⁺) as etching ion species and to set an etching speed (etching rate) as 0.05 nm/sec (a conversion value for an SiO₂ thermally-oxidized film).

Moreover, from the viewpoint of forming a gas barrier layer having a uniform surface and excellent gas barrier properties all over the surface of the first barrier layer, it is preferable that the first gas barrier layer is substantially uniform in terms of the component in the surface direction (the direction parallel to the surface of the first gas barrier layer).

The first gas barrier layer being substantially uniform in the surface direction means that, when the distribution curve of oxygen and the distribution curve of carbon are produced as to any two measurement points on the surface of the first gas barrier layer obtained by the XPS depth profile measurement, the carbon distribution curves for the arbitrary two measurement points have the same number of extreme values, and that the absolute values of the differences between the largest value and the smallest value of the atomic ratio of carbon of the respective carbon distribution curves are the same as each other or have a difference within 5 at %.

The gas barrier layer 12 preferably includes at least one first gas barrier layer that satisfies all of the conditions (i) to (iv) described above, and may include two or more layers that satisfy the requirements.

In a case where two or more first gas barrier layers are provided, the plural first gas barrier layers may be composed of the same material or different materials. In addition, the gas barrier layer 12 described above may be formed on one surface of the substrate 11 or on both surfaces of the substrate 11.

Furthermore, in distribution curve of silicon, the distribution curve of oxygen, and the distribution curve of carbon, in a case where the atomic ratio of silicon, the atomic ratio of oxygen, and the atomic ratio of carbon satisfy the condition represented by the above condition (i) in the region corresponding to 90% or more of the thickness of the first gas barrier layer, the atomic ratio of silicon in the first gas barrier layer is preferably within the range of 25 to 45 at %, more preferably within the range of 30 to 40 at %.

Additionally, the atomic ratio of oxygen in the first gas barrier layer is preferably within the range of 33 to 67 at %, more preferably within the range of 45 to 67 at %.

Moreover, the atomic ratio of carbon in the first gas barrier layer is preferably within the range of 3 to 33 at %, more preferably within the range of 3 to 25 at %.

(Thickness of First Gas Barrier Layer)

The thickness of the first gas barrier layer is preferably within the range of 5 to 3000 nm, more preferably within the range of 10 to 2000 nm, furthermore preferably within the range of 100 to 1000 nm, and particularly preferably within the range of 300 to 1000 nm. When the thickness of the first gas barrier layer is within the above range, the first gas barrier layer is excellent in gas barrier properties such as an oxygen gas barrier property and a water vapor barrier property, and does not lower the gas barrier properties even after bending.

(Method for Forming First Gas Barrier Layer)

The first gas barrier layer is preferably a layer formed by the plasma enhanced chemical vapor deposition. More specifically, the first gas barrier layer formed by the plasma enhanced chemical vapor deposition is formed by the plasma enhanced chemical vapor deposition in which the substrate 11 is conveyed according to a roll-to-roll system while being in contact with a pair of film-deposition rollers and plasma is discharged while a film-deposition gas is supplied between the film-deposition rollers.

Furthermore, during the discharge between the pair of film-deposition rollers, it is preferable that the polarity of the pair of film-deposition rollers is alternately inverted. The film-deposition gas used in the plasma enhanced chemical vapor deposition preferably includes an organosilicon compound and oxygen. The content of the oxygen in the film-deposition gas to be supplied is preferably equal to or less than a theoretical oxygen quantity required for the complete oxidation of the entire quantity of the organosilicon compound in the film-deposition gas. Furthermore, the first gas barrier layer is preferably a layer formed on the substrate 11 by a continuous film-deposition process. The plasma enhanced chemical vapor deposition may be the plasma enhanced chemical vapor deposition of the Penning discharge plasma system.

In order to forma layer in which an atomic ratio of carbon has a concentration gradient and continuously varies in the layer just like the first gas barrier layer, it is preferable to generate plasma discharge in the space between a plurality of the film-deposition rollers when generating the plasma in the plasma enhanced chemical vapor deposition.

Moreover, it is preferable to use the pair of the film-deposition rollers, to convey the pair of the film-deposition rollers while bringing the substrate 11 into contact with each of the pair of the film-deposition rollers, and to generate plasma by an electric discharge in the space between the pair of the film-deposition rollers. The distance between the substrate 11 and the position of the plasma discharge between the film-deposition rollers varies, by using the pair of the film-deposition rollers, by conveying the pair of the film-deposition rollers while bringing the substrate 11 into with each of the pair of the film-deposition rollers, and by discharging plasma between the pair of the film-deposition rollers, and thus it becomes possible to form the gas barrier layer in which an atomic ratio of carbon has a concentration gradient and continuously varies in the layer.

In addition, since it becomes possible, at the film formation, to perform film-formation of a surface portion of the substrate 11 existing on one film-deposition roller, and at the same time, to also perform film-formation of a surface portion of the substrate 11 existing on another film-deposition roller, the film formation can be efficiently achieved, and a film-forming rate can be increased twice. Furthermore, since it is possible to form the film having the same configuration, the extreme values in the carbon distribution curves can at least be doubled, and the gas barrier layers that satisfy all of the above conditions (i) to (iv) can be efficiently formed.

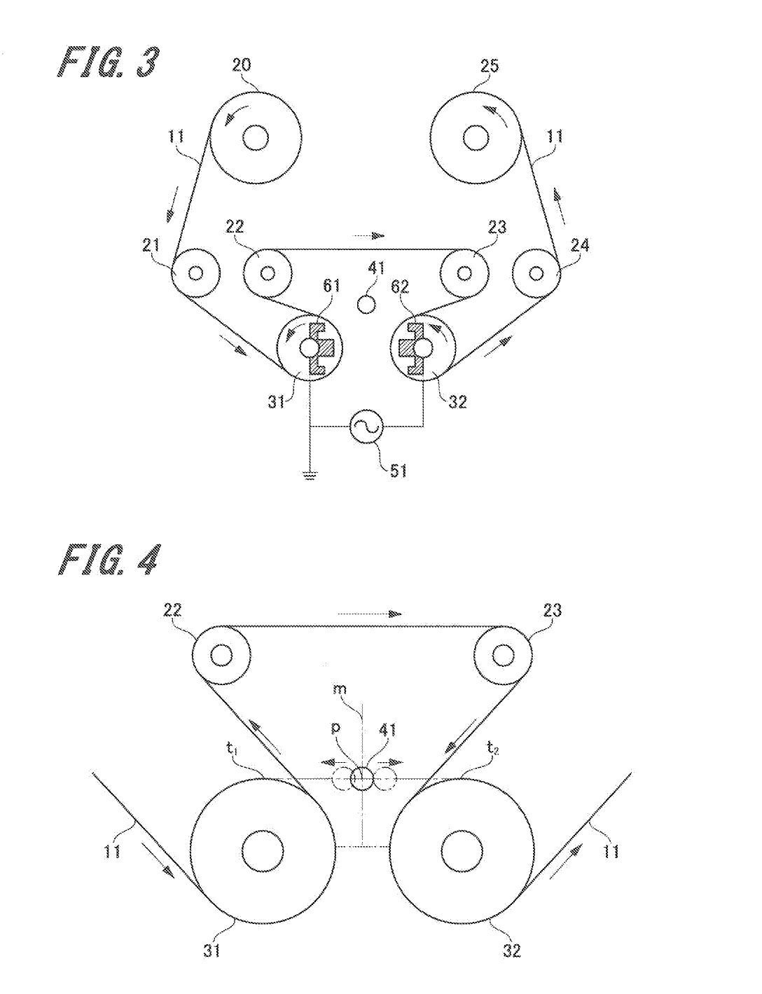

In addition, any apparatus that can be used for the manufacturing of the gas barrier film by the plasma enhanced chemical vapor deposition is not particularly limited, but it is preferable that the apparatus has a configuration of including at least a pair of film-deposition rollers and a plasma power source, and of being capable of discharging electricity in the space between the pair of the film-deposition rollers. For example, it becomes possible to form the gas barrier film by using the roll-to-roll system while utilizing the plasma enhanced chemical vapor deposition, by employing the manufacturing apparatus illustrated in FIG. 3 as described below.

Hereinafter, a method for forming the first gas barrier layer will be explained in detail by referring to FIG. 3. Note that FIG. 3 is a schematic view showing one example of the manufacturing apparatus that can be preferably utilized for forming the first gas barrier layer on the substrate 11.

(Manufacturing Apparatus)

The manufacturing apparatus shown in FIG. 3 includes a delivery roller 20, conveyance rollers 21, 22, 23, and 24, film-deposition rollers 31 and 32, a gas inlet 41, a power source 51 for plasma generation, magnetic-field generators 61 and 62 disposed inside the film-deposition rollers 31 and 32, and a winding roller 25.

In addition, the manufacturing apparatus shown in FIG. 3 includes a vacuum chamber (not shown) that disposes at least the film-deposition rollers 31 and 32, the gas inlet 41, the power source 51 for plasma generation, and the magnetic-field generators 61 and 62 made of permanent magnets. Moreover, in the manufacturing apparatus described above, the vacuum chamber is connected to a vacuum pump (not shown), and the vacuum pump can appropriately adjust the pressure in the vacuum chamber.

Furthermore, each of the film-deposition rollers is connected to the power source 51 for plasma generation in order that the pair of the film-deposition rollers (film-deposition roller 31 and film-deposition roller 32) can function as a pair of counter electrodes. Accordingly, it becomes possible to discharge electricity in the space between the film-deposition roller 31 and the film-deposition roller 32, from the film-deposition roller 31 and the film-deposition roller 32, by electric power supplied from the power source 51 for plasma generation. Thereby, it is possible to generate plasma in the space between the film-deposition roller 31 and the film-deposition roller 32.

Note that, when the film-deposition roller 31 and the film-deposition roller 32 are utilized as electrodes, the material and design thereof are appropriately modified as to be capable of being used as electrodes. Furthermore, the pair of the film-deposition rollers 31 and 32 are preferably disposed such that the central axes of the rollers are substantially parallel to each other on the same plane. Such an arrangement of the pair of the film-deposition rollers 31 and 32 doubles the deposition rate and can at least double the number of extreme values in the carbon distribution curve because a film having an identical structure is formed.

Moreover, the magnetic-field generators 61 and 62 are provided inside the film-deposition roller 31 and the film-deposition roller 32. The magnetic-field generators 61 and 62 are provided to be fixed without rotating the magnetic-field generators 61 and 62 themselves even when the film-deposition rollers 31 and 32 rotate.

Known rollers may be appropriately used as the film-deposition roller 31 and the film-deposition roller 32. From the viewpoint of efficiently forming a thin film, the film-deposition rollers 31 and 32 having the same diameter are preferably used. Furthermore, the diameter of such film-deposition rollers 31 and 32 is preferably within the range of 300 to 1000 mmφ, more preferably within the range of 300 to 700 mmφ, from the viewpoint of the discharge conditions and the space in the chamber, and the like. When the diameter is more than 300 mmφ, the productivity is not so lowered since the plasma discharge space is not decreased. In addition, it is possible to reduce damage to the substrate 11 since the total heat due to the plasma discharge can be prevented from being applied to the film in a short time. On the other hand, when the diameter is less than 1000 mmφ, it is possible to maintain practicality in a device design including uniformity of a plasma discharge space.

In addition, known rollers can be appropriately used as the delivery roller 20 and the conveyance rollers 21, 22, 23, and 24. Furthermore, the winding roller 25 is not particularly limited as long as the substrate 11 (gas barrier film) on which the first gas barrier layer is formed can be wound, and known rollers can be appropriately used as the winding roller 25.

An inlet that can supply or discharge a raw material gas or the like at a predetermined rate can be appropriately used as the gas inlet 41.

Known magnetic-field generators can be appropriately used as the magnetic-field generators 61 and 62.

A power source for a known plasma generator can be appropriately used as the power source 51 for plasma generation. The power source 51 for plasma generation as described above supplies power to the film-deposition roller 31 and the film-deposition roller 32 which are connected thereto, and thus it becomes possible to utilize the film-deposition roller 31 and the film-deposition roller 32 as the counter electrodes for electric discharge.

It becomes possible to efficiently perform plasma CVD method as the power source 51 for plasma generation, and thus it is preferable to utilize a source (AC source, or the like) that can alternatively invert polarities of the pair of the film-deposition rollers. Furthermore, it is more preferable that the power source 51 for plasma generation can apply power within the range of 100 W to 10 kW and an AC frequency can be within the range of 50 Hz to 500 kHz.

(Manufacturing Method of Gas Barrier Layer)

Through the use of the manufacturing apparatus shown in FIG. 3, the first gas barrier layer can be manufactured by the plasma CVD method by appropriately adjusting, for example, a type of raw material gas, electric power of an electrode drum in a plasma generator, pressure in a vacuum chamber, a diameter of a film-deposition roller, and conveying rate of the substrate 11.

Namely, through the use of the manufacturing apparatus shown in FIG. 3, the first gas barrier layers can be formed on the surface of the substrate 11 on the film-deposition roller 31 and on the surface of the substrate 11 on the film-deposition roller 32 by generating the plasma discharge between the pair of the film-deposition rollers (film-deposition rollers 31, 32) while supplying a film-deposition gas (raw material gas, or the like) into the vacuum chamber, to thereby decompose the film-deposition gas (raw material gas, or the like) by plasma.

Note that, in such film-deposition, the substrate 11 is conveyed by the delivery roller 20 and the film-deposition roller 31, and the like, and the first gas barrier layer is formed on the surface of the substrate 11 through the continuous film-deposition process of the roll-to-roll system.

In the first gas barrier layer, it is preferable that the maximum value of the distribution curve of oxygen closest to the surface of the first gas barrier layer on the substrate 11 side is the largest value of the maximum values of the oxygen distribution curve of the first gas barrier layer so that the distribution curve of oxygen satisfies the above-described condition (iv).

Furthermore, it is preferable, as the atomic ratio of oxygen, that the atomic ratio of oxygen serving as the maximum value of the distribution curve of oxygen closest to the surface of the first gas barrier layer on the substrate 11 side is preferably 1.05 times or more the atomic ratio of oxygen serving as the maximum value of the distribution curve of oxygen closest to the surface of the second gas barrier layer opposite to the substrate 11 side.

As described above, the method for forming the first gas barrier layer so that atomic ratio of oxygen has a predetermined distribution in the first gas barrier layer is not particularly limited, and there can be used a method for varying the concentration of the film-deposition gas during deposition, a method for changing the position of the gas inlet 41, a method for performing gas supply at multiple positions, a method for controlling the flow of the gas by disposing a shielding plate or the like near the gas inlet 41, and a method for performing plasma CVD multiple times by changing concentrations of the film-deposition gas, and the like. Among them, a method for performing plasma CVD film-deposition by changing the position of the gas inlet 41 near either of the film-deposition roller 31 or the film-deposition roller 32 is preferable because the method is simple and has good reproducibility.

(Gas Inlet)

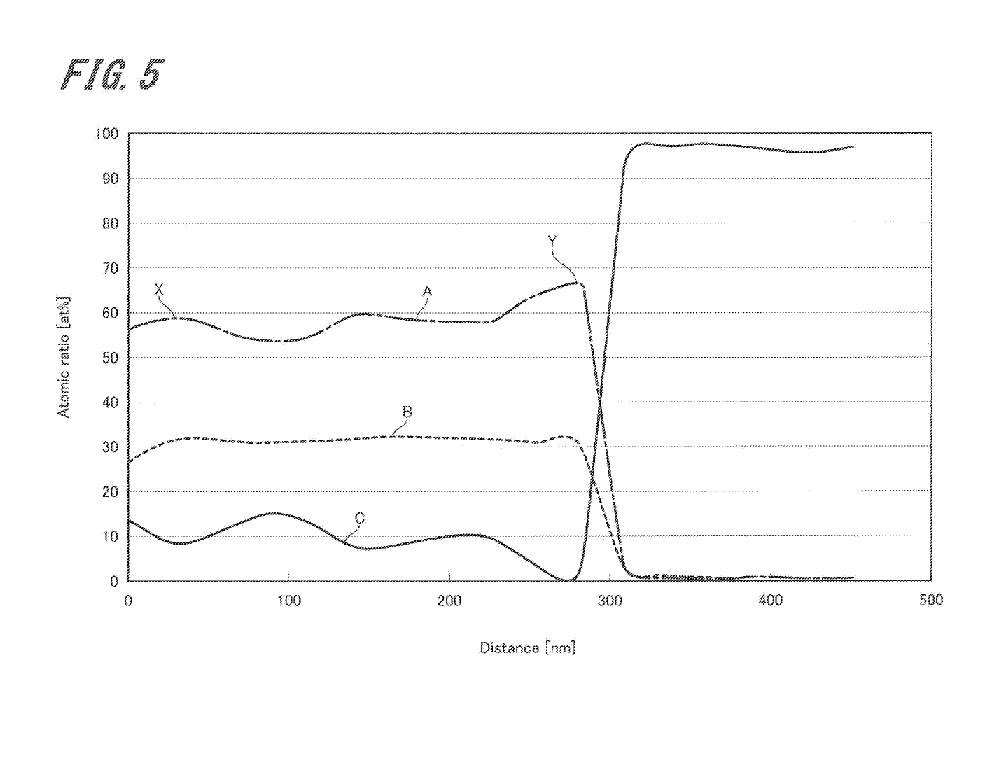

FIG. 4 is a schematic view for explaining a moving state of the position of the gas inlet 41 in the manufacturing apparatus shown in FIG. 3.

In the oxygen distribution curve of the first gas barrier layer, when the distance between the gas inlet 41 and the film-deposition roller 31 or the film-deposition roller 32 is set to be 100%, the gas inlet 41 is moved toward the film-deposition roller 31 or the film-deposition roller 32 within the range of 5 to 20% from the perpendicular bisector m of the line segment connecting the film-deposition roller 31 and the film-deposition roller 32 to thereby be able to perform control as to satisfy the condition of the extreme value.

Namely, when the distance t₁p or the distance t₂p is set to be 100% from the point p on the perpendicular bisector m of the line segment connecting the film-deposition roller 31 and the film-deposition roller 32 in the direction of the point t₁ of the film-deposition roller 31 or the point t₂ of the film-deposition roller 32, the gas inlet 41 is moved parallel toward the point t₁ of the film-deposition roller 31 or the point t₂ of the film-deposition roller 32 within the range of 5 to 20% from the position of the point p.

In this case, the value of the extreme value of the oxygen distribution curve can be controlled by the distance of the movement of the gas inlet 41. For example, in order to increase the extreme value of the oxygen distribution curve of the first gas barrier layer, the gas inlet 41 is moved closer to the point t₁ of the film-deposition roller 31 or the point t₂ of the film-deposition roller 32. It is possible to increase the atomic ratio of oxygen of the first gas barrier layer by making the distance between the gas inlet 41 and the film-deposition roller 31 or the film-deposition roller 32 smaller. Moreover, it is possible to decrease the atomic ratio of oxygen of the first gas barrier layer by making the distance between the gas inlet 41 and the film-deposition roller 31 or the film-deposition roller 32 larger.

The moving range of the gas inlet 41 is preferably within the range of 5 to 20%, more preferably 5 to 15%. When the movement is within the above range, there is not generated unevenness in the oxygen distribution curve and other element distribution curves in the surface, and it is possible to uniformly reproduce well the predetermined distribution.

(Element Profile)

Each element profile shown in the above FIG. 2 is the XPS depth profile of the layer which is formed by moving the gas inlet 41 closer to the direction of the film-deposition roller 31 by 5% in the first gas barrier layer.

Furthermore, FIG. 5 shows an example of the profile of each element in the thickness direction obtained by the XPS depth profile of the layer which is formed by moving the gas inlet 41 closer to the direction of the film-deposition roller 32 by 10%.

In both FIG. 2 and FIG. 5, when X is a maximum value of the atomic ratio of oxygen closest to the surface of the first gas barrier layer on the oxygen distribution curve A, and Y is a maximum value of the atomic ratio of oxygen closest to the surface of the substrate 11, the relation of Y>X is satisfied.

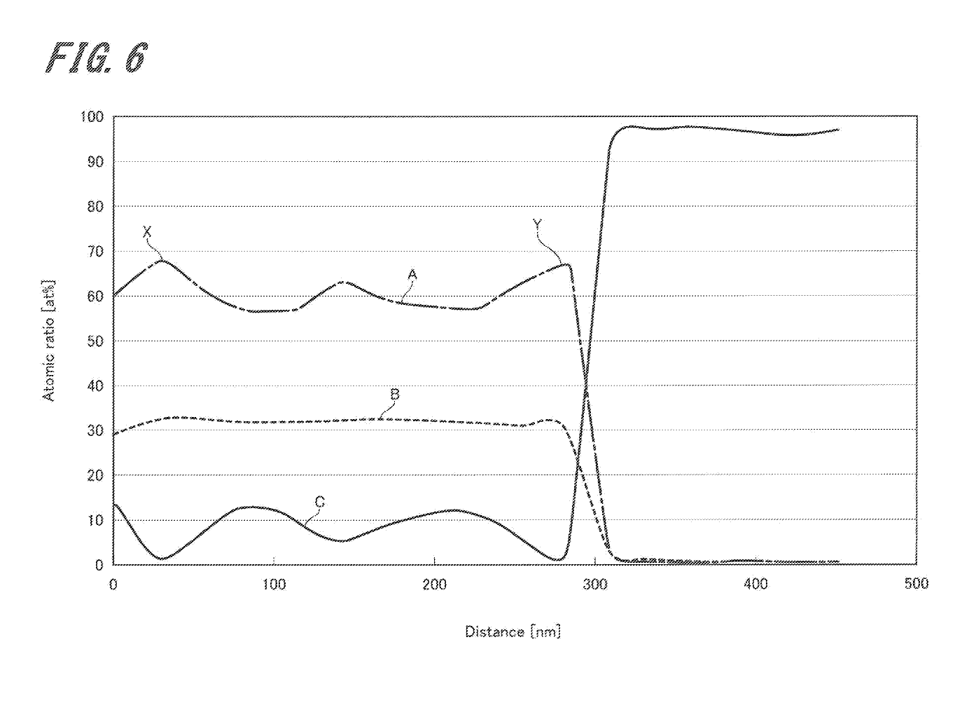

On the other hand, FIG. 6 shows, as a comparison, the profile of each element obtained by the XPS depth profile of the gas barrier layer which is formed by disposing the gas inlet 41 on the perpendicular bisector m of the line segment connecting the film-deposition rollers 31 and 32.

As shown in FIG. 6, X which is the atomic ratio of oxygen serving as a maximum value of the distribution curve of oxygen on the surface of the gas barrier layer closest to the substrate 11 side is almost similar to Y which is the atomic ratio of oxygen serving as a maximum value of the distribution curve of oxygen closest to the surface of the gas barrier layer which is opposite side of the substrate 11. Accordingly, it is found that, when the gas inlet 41 is forming by disposing the inlet on the perpendicular bisector m of the line segment connecting the film-deposition rollers 31 and 32, an extreme value of the distribution curve of oxygen on the surface of the gas barrier layer closest to the substrate 11 side does not serve as the maximum value in the layer.

(Raw Material Gas)

The raw material gas in the film-deposition gas used for forming the first gas barrier layer can be appropriately selected depending on the material of the gas barrier layer to be formed. For example, organosilicon compounds containing silicon can be preferably used as the raw material gas described above.

Examples of the organosilicon compounds include hexamethyldisiloxane, 1,1,3,3-tetramethyldisiloxane, vinyltrimethylsilane, methyltrimethylsilane, hexamethyldisilane, methylsilane, dimethylsilane, trimethylsilane, diethylsilane, propylsilane, phenylsilane, vinyltriethoxysilane, vinyltrimethoxysilane, tetramethoxysilane, tetraethoxysilane, phenyltrimethoxysilane, methyltriethoxysilane, octamethylcyclotetrasiloxane, and the like.

Among these organosilicon compounds, hexamethyldisiloxane and 1,1,3,3-tetramethyldisiloxane are preferred from the viewpoint of the handling during film deposition and of properties such as the gas barrier properties of the resulting first gas barrier layer. Furthermore, these organosilicon compounds can be used alone or in combination of two or more kinds.

Furthermore, a reactive gas in addition to the raw material gas may be used together as the film-deposition gas. The above-described reactive gas can be appropriately selected, for use, from gases that produce inorganic compounds such as oxides and nitrides by reaction with the raw material gas.

The reactive gas for the formation of the oxides which can be used includes, for example, oxygen and ozone. Further, the reactive gas for the formation of the nitrides which can be used includes, for example, nitrogen and ammonia.

These reactive gases can be used alone or in combination of two or more kinds. For example, in a case of forming an oxide nitride compound, the reactive gas for the formation of the oxides can be combined for use with the reactive gas for the formation of the nitrides.

A carrier gas may also be used as the film-deposition gas, as necessary, for supplying the raw material gas to the vacuum chamber. Furthermore, a discharge gas may also be used as the film-deposition gas, as necessary, for generating the plasma discharge. A known gas can be appropriately used as the carrier gas and the discharge gas as described above, and examples that can be used include a rare gas such as helium, argon, neon, or xenon.

When the film-deposition gas contains the raw material gas and the reactive gas, it is preferable to include the reactive gas at a percentage not excessively higher than the percentage of the reactive gas theoretically required for complete reaction of the raw material gas and the reactive gas. When the percentage of the reactive gas is excessively high, the required first gas barrier layer is hard to be obtained. Therefore, in order to obtain required performance as the barrier film, it is preferable that, for example, when the film-deposition gas contains the organosilicon compound and oxygen, the amount of oxygen is equal to or less than a theoretical amount of oxygen required for complete oxidation of all of the organosilicon compounds in the film-deposition gas.

Hereinafter, explanation will be made by using, as the representative example, hexamethyldisiloxane (organosilicon compound: HMDSO, (CH₃)₆Si₂O) as the raw material gas and oxygen (O₂) as the reactive gas.

In a case where a silicon-oxygen thin film is produced by reaction, through the plasma CVD method, of the film-deposition gas containing hexamethyldisiloxane (HMDSO: (CH₃)₆Si₂O) as the raw material gas and oxygen (O₂) as the reactive gas, the reaction shown in the following reaction formula (1) takes place to thereby manufacture silicon oxide.

(CH₃)₆Si₂O+12O₂→6CO₂+9H₂O+2SiO₂ (1)

In the reaction like this, 12 moles of oxygen is required for complete oxidation of 1 mole of hexamethyldisiloxane. Accordingly, in a case where 12 moles or more oxygen is contained for each one mole of hexamethyldisiloxane for the complete reaction in the film-deposition gas, a uniform silicon dioxide film is formed. Thereby, the adjustment of the flow ratio of the reaction gas is performed so as to be a ratio equal to or less than the theoretical ratio for a complete reaction to thereby promote an incomplete reaction. Namely, the amount of oxygen is required to be less than 12 moles that is the stoichiometric ratio relative to 1 mole of hexamethyldisiloxane.

Note that, in the reaction within the actual plasma CVD chamber, hexamethyldisiloxane as the raw material gas and oxygen as the reactive gas are supplied from the gas inlets to the film-deposition region, for film deposition, and thus, even if the molar amount (flow rate) of oxygen of the reactive gas is 12 times molar amount (flow rate) larger than the molar amount (flow rate) of hexamethyldisiloxane of the raw material gas, the reaction actually cannot be completely made to progress, and the c reaction is considered to be completed only when oxygen is supplied in a significantly excessive amount to the stoichiometric ratio. For example, there is a case where the molar amount (flow rate) of oxygen is set to at least approximately 20 times larger than the molar amount (flow rate) of hexamethyldisiloxane of the raw material gas in order to complete the oxidizing reaction to thereby obtain silicon oxide by the CVD method. Accordingly, the mole amount (flow rate) of oxygen of the reaction gas relative to the molar amount (flow rate) of hexamethyldisiloxane of the raw material gas is preferably 12 times or less that is the stoichiometric ratio, more preferably 10 times or less.

When hexamethyldisiloxane and oxygen are contained in such ratios, the carbon atoms and hydrogen atoms in the hexamethyldisiloxane not completely oxidized are incorporated into the first gas barrier layer to thereby form the desired first gas barrier layer. Accordingly, it becomes possible to achieve excellent barrier properties and bending resistance.

Furthermore, the lower limit of the molar amount (flow rate) of oxygen relative to the molar amount (flow rate) of hexamethyldisiloxane in the film-deposition gas is preferably more than 0.1 times of the molar amount (flow rate) of hexamethyldisiloxane, more preferably more than 0.5 times.

(Vacuum Level)

The pressure (vacuum level) in the vacuum chamber can be appropriately adjusted depending on the kind of raw material gas and the like and is preferably within the range of 0.5 to 100 Pa.

(Roller Deposition)

In such a plasma CVD method, in order to discharge electricity between the film-deposition rollers 31 and 32, the electric power to be applied to electrode drums connected to the power source 51 for plasma generation and disposed on the film-deposition rollers 31 and 32 can be appropriately adjusted depending on the kind of the raw material gas, the pressure in the vacuum chamber, and the like. For example, the electric power is preferably within the range of 0.1 to 10 kW.

When the electric power applied is within the above range, generation amount of particles is small, and the heat generated during film deposition is within control. Thus, there are also no thermal damage in the substrate 11 and generation of wrinkles at the time of film deposition, due to the increase in temperature on the surface of the substrate 11 during film deposition. Furthermore, there is small possibility that the film-deposition rollers themselves are damaged, because of the melting of the substrate 11 by heating and the discharge of a large current between the naked film-deposition rollers.

The conveying rate (line speed) of the substrate 11 can be appropriately adjusted depending on the kind of the raw material gas and the pressure in the vacuum chamber, and the like, but is preferably within the range of 0.25 to 100 m/min, more preferably within the range of 0.5 to 20 m/min. When the line rate is within the above range, wrinkles in the substrate 11 due to heat are not easily generated, and the thickness of the first gas barrier layer to be formed can also be sufficiently controlled.

(Second Gas Barrier Layer)

With respect to the gas barrier layer 12, there is preferably provided, on the first gas barrier layer, the second gas barrier layer in which a coated film by a solution containing polysilazane is subjected to modification treatment by irradiation with a vacuum ultraviolet rays (VUV rays) having a wavelength of 200 nm or less. When the second gas barrier layer is provided on the first gas barrier layer provided by the plasma CVD method, it is possible to bury the polysilazane gas barrier component from the upper part, in minute defects remaining on the first gas barrier layer. Accordingly, it is possible to further enhance the gas barrier properties and bending properties of the gas barrier layer 12.

The second gas barrier layer preferably has a thickness within the range of 1 to 500 nm, more preferably within the range of 10 to 300 nm. When the thickness is more than 1 nm, the gas barrier properties can be exhibited, and when the thickness is within the range of 500 nm or less, cracks are not easily generated in the dense silicon oxide film.

(Polysilazane)

In the second gas barrier layer, the polysilazane represented by the following General formula (A) can be used.

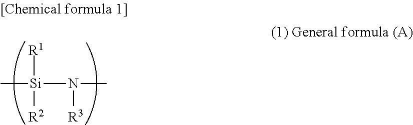

In the Formula 1, R¹, R² and R³ each represent a hydrogen atom, an alkyl group, an alkenyl group, a cycloalkyl group, an aryl group, an alkylsilyl group, an alkylamino group, or an alkoxy group.

The polysilazane where R¹, R² and R³ in the General formula (A) are all hydrogen atoms, namely, perhydropolysilazane (PHPS) is particularly preferable from the viewpoint of the denseness of the obtained second gas barrier layer. The perhydropolysilazane is presumed to have a linear chain structure and a cyclic structure centering around 6- and 8-membered rings. A number average molecular weight (Mn) thereof is about 600 to 2000 (in terms of polystyrene by gel permeation chromatography), and is in the form of liquid or solid.

The second gas barrier layer can be formed by coating the coating solution containing polysilazane on the first gas barrier layer by the CVD method, by drying the coated solution, and then by performing irradiation with the vacuum ultraviolet.

It is preferable to evade, as an organic solvent used for the preparation of a polysilazane coating solution, a solvent that does not contain a lower alcohol or water, which easily reacts with polysilazane. Examples that can be used include hydrocarbon solvents such as an aliphatic hydrocarbon, an alicyclic hydrocarbon, and an aromatic hydrocarbon; halogenated hydrocarbon solvents; and ethers such as an aliphatic ether and an alicyclic ether. Specific examples that can be used include hydrocarbons such as pentane, hexane, cyclohexane, toluene, xylene, Solvesso, and turpentine; halogenated hydrocarbons such as methylene chloride and trichloroethane; ethers such as dibutyl ether, dioxane, and tetrahydrofuran, and the like. These organic solvents may be selected in accordance with purposes such as the solubility of polysilazane and evaporation rate of the solvent, and may also be used by mixture of a plurality of the organic solvents.

The concentration of polysilazane in the coating solution containing polysilazane is different depending on the thickness of the second gas barrier layer and the pot life of the coating solution, and is preferably approximately 0.2 to 35% by mass.

In order to promote the modification to the silicon oxide nitride, an amine catalyst or a metal catalyst such as a Pt compound such as Pt acetylacetonate, a Pd compound such as propionic acid Pd, or an Rh compound such as Rh acetylacetonate can also be added to the coating solution. The use of the amine catalyst is particularly preferable. Examples of the specific amine catalyst include N,N-diethylethanolamine, N,N-dimethylethanolamine, triethanolamine, triethylamine, 3-morpholinopropylamine, N,N,N′,N′-tetramethyl-1,3-diaminopropane, N,N,N′,N′-tetramethyl-1,6-diaminohexane, and the like.

The catalyst amount to be added to polysilazane is preferably within the range of 0.1 to 10% by mass, more preferably within the range of 0.2 to 5% by mass, and further preferably within the range of 0.5 to 2% by mass relative to the total amount of the coating solution. When the amount to be added of the catalyst is within the above range, it is possible to evade the excessive silanol formation due to a rapid progress of a reaction, a decrease in the film density, an increase in film defects, and the like.

An arbitrary and appropriate method can adopted as a coating method of the coating solution containing polysilazane, and specific examples thereof included roll coating, flow coating, inkjet printing, spray coating, printing, dip coating, casting, bar coating, gravure printing, and the like.

The thickness of the coating film can be appropriately determined depending on the intended purpose. For example, the thickness of the coating film is preferably within the range of 50 nm to 2 μm, more preferably within the range of 70 nm to 1.5 μm, and further preferably within the range of 100 nm to 1 μm.

(Excimer Treatment)

In the second gas barrier layer, at least a part of the polysilazane is modified to silicon oxide nitride in the process of irradiating the coating film containing the polysilazane with the vacuum ultraviolet ray.

Hereinafter, the presumed mechanism in which the coating film containing the polysilazane in the process of the vacuum ultraviolet ray irradiation is modified into the specific composition SiOₓN_{y} will be explained by taking perhydropolysilazane as an example.

Perhydropolysilazane can be represented by the composition [—(SiH₂—NH)ₙ—]. When represented by SiOₓN_{y}, x=0 and y=1. An external oxygen source is necessary to achieve x>0. Examples of such external oxygen sources include the following (i) to (v).

(i) Oxygen and water contained in the polysilazane coating solution; (ii) Oxygen and water absorbed in the coating film from the atmosphere during application and drying; (iii) Oxygen, water, ozone, and singlet oxygen absorbed in the coating film from the atmosphere during the vacuum ultraviolet ray irradiation; (iv) Oxygen and water outgassed from the substrate side and migrated into the coating film due to heat and other factors applied during the vacuum ultraviolet ray irradiation; (v) Oxygen and water absorbed by the coating film from an oxidizing atmosphere when the film is moved from a non-oxidizing atmosphere, where vacuum ultraviolet ray irradiation is performed, to the oxidizing atmosphere.

On the other hand, the upper limit of y is basically 1, because the nitridation of Si atoms is seemed to be very rare compared with the oxidation thereof.

Basically, x and y are within the range defined by 2x+3y≦4 on the basis of the number of valence electrons in Si, O, and N atoms. When the state of y=0 after completing the oxidation, the coating film contains silanol groups, and there is the case where the range is 2<x<2.5.

Hereinafter, there will be explained the reaction mechanism presumed to generate silicon oxide nitride and further silicon oxide from the perhydropolysilazane, in the vacuum ultraviolet ray irradiation process.

(1) Dehydrogenation and Formation of Si—N Bond Along with that

It is considered that the Si—H bond and the N—H bond in the perhydropolysilazane are relatively and easily cleaved due to the excitation induced by the vacuum ultraviolet ray irradiation or the like, and are recombined to the Si—N bond under an inert atmosphere. Furthermore, there is a case where a dangling bond of Si atom may also be formed. Namely, the film is cured as the composition of SiN_{y} without oxidation. In this case, the cleavage of a polymer main chain does not occur. The cleavage of the Si—H bond and the N—H bond is accelerated by a catalyst and by heating. The thus cleaved hydrogen is released in the form of H₂ from the film to the outside the film.

(2) Formation of Si—O—Si Bond Due to Hydrolysis and Dehydration Condensation

The Si—N bond in the perhydropolysilazane is hydrolyzed to cleave the polymer main chain and to produce a Si—OH. Two Si—OHs are condensed by dehydration into a Si—O—Si bond to be cured. Though such a reaction also occurs in the atmosphere, the main water source during the vacuum ultraviolet ray irradiation under an inert atmosphere is probably water vapor outgassed from the substrate due to the heat generated during the irradiation. Excess water causes some Si—OHs to remain without dehydration condensation, and thus, a cured film having a composition SiO_{2.1 to 2.3} has a poor gas barrier property.

(3) Formation of Si—O—Si Bond Involving Direct Oxidation by Singlet Oxygen

An appropriate amount of oxygen in the atmosphere during the vacuum ultraviolet ray irradiation forms highly oxidative singlet oxygen (O). The H and N atoms in the perhydropolysilazane are replaced with O atom to form Si—O—Si bond to cure the film. It is presumed that there is a case where the cleavage of the polymer main chain may also cause recombination of the bonds.

(4) Oxidation Accompanying Cleavage of Si—N Bond Due to Vacuum Ultraviolet Ray Irradiation and Excitation

It is presumed that since the energy of the vacuum ultraviolet rays is higher than the energy of the Si—N bond in the perhydropolysilazane, the Si—N bond is cleaved, and at this time, when there is an oxygen source such as oxygen, ozone, water, or the like, in the environment, the cleaved Si is oxidized to form a Si—O—Si bond or a Si—O—N bond. It is also presumed that there is a case where the recombination of the bonds may be yielded by the cleavage of the polymer main chain.

The composition of silicon oxide nitride in the coating film containing the polysilazane can be adjusted by controlling the oxidized level through an appropriate combination of the above-described oxidation mechanisms (1) to (4).

In the vacuum ultraviolet ray irradiation process, the illuminance of the vacuum ultraviolet rayon the surface of the polysilazane-containing coating film is preferably within the range of 30 to 200 mW/cm², more preferably within the range of 50 to 160 mW/cm². When the illuminance is 30 mW/cm² or more, there is no concern that modification efficiency may be lowered, and when the illuminance is 200 mW/cm² or less, ablation of the coating film is not generated and damage to the substrate 11 is small.

An amount of the irradiation energy of the vacuum ultraviolet ray on the surface of the polysilazane-containing coating film is preferably within the range of 200 to 10000 mJ/cm², more preferably within the range of 500 to 5000 mJ/cm². When the amount is 200 mJ/cm² or more, sufficient modification can be carried out, and when the amount is 10000 mJ/cm² or less, excessive modification is not achieved, and thus cracking and thermal deformation of the substrate 11 are small.

(Vacuum Ultraviolet Ray Irradiation Apparatus Having Excimer Lamp)

Example of the ultraviolet ray irradiation apparatus includes a rare gas excimer lamp which emits a vacuum ultraviolet ray having a wavelength within the range of 100 to 230 nm.

Since the atom of a rare gas such as xenon (Xe), krypton (Kr), argon (Ar) or neon (Ne) does not produce a molecule by chemical bonding, the rare gas is referred to as an inert gas. However, the atom of the rare gas energized by electric discharge (excited atom) can bond with other atoms to thereby produce a molecule.

In a case where the rare gas is Xe (xenon), as shown in the following reaction formula, when the excited excimer molecule Xe₂* transits to the ground state, 172 nm excimer light is emitted.

e+Xe→Xe*

Xe*+2Xe→Xe₂*+Xe

Xe₂*→Xe+Xe+hν(172 nm)

The feature of the excimer lamp is high efficiency since the radiation of light is concentrated on a single wavelength and there is almost no radiation of light except for necessary light. Moreover, the temperature of the target can be maintained at a relatively low level because excessive light is not radiated. Furthermore, the excimer lamp can be turned on/off instantaneously since it does not take time to start or restart the lamp.

Alight source that efficiently performs irradiation with the excimer light efficiently includes a dielectric-barrier discharge lamp.

The dielectric-barrier discharge lamp may be generally configured by disposing at least one electrode in a discharge reservoir made of a dielectric material and at an outside thereof so as to generate the electric discharge between the electrodes via the dielectric material. For example, there is a configuration in which a rare gas such as xenon gas is sealed in a double-cylinder-type electric discharge reservoir formed of a big tube and a fine tube made of quartz glass, and a mesh first electrode is attached on the outside of the discharge reservoir and further the other electrode is provided inside of the inner tube. In the dielectric-barrier discharge lamp, by applying a high-frequency voltage between the electrodes, a dielectric-barrier discharge is generated in the discharge reservoir. In addition, at the time when the excimer molecule such as xenon yielded at the discharge is disassociated, the excimer light is generated.

The dielectric-barrier discharge is a significantly narrow micro-discharge, similar to thunder, that is generated in a gas space in response to the application of a high-frequency high-voltage of several tens of kilohertz to electrodes, the gas space being disposed between the electrodes through dielectric substance, such as transparent quartz. When the micro-discharge streamer reaches the tube wall (dielectric material), the electric charges are stored on the surface of the dielectric, and thus, the micro-discharge disappears. The dielectric-barrier discharge is a discharge where the micro-discharges are spread over the entire tube wall, and the cycles of generation and disappearance are repeated. Thus, the flickering of light can be visually confirmed. Furthermore, since streamers of an extremely high temperature directly reach local points of the tube wall, there is a possibility that degradation of the tube wall is accelerated.

Other than the dielectric-barrier discharge, an electrodeless field discharge is also means for generating the excimer emission efficiently. The electrodeless field discharge occurs as a result of capacitive coupling and is also referred to as RF discharge. The lamp, the electrodes, and their arrangement are basically the same as those for the dielectric-barrier discharge. The high frequency applied to the electrodes illuminates the lamp at several MHz. Such spatially or temporally uniform discharge achieved through electrodeless field discharge provides a lamp having a long life without flickering.

Since, in the dielectric-barrier discharge, the micro-discharge is generated only between the electrodes, in order to discharge over the entire discharge space, it is necessary to cover the entire external surface with an external electrode, and the electrode should transmit the light for taking out the light to the outside.

Thus, a mesh of thin metal wires is used as the electrode. The electrode is composed of very thin wires that do not block light. Unfortunately, the electrode is easy to be damaged in an oxygen atmosphere by ozone generated by the vacuum ultraviolet rays. This can only be avoided by providing an inert gas atmosphere such as a nitrogen atmosphere, around the lamp inside the irradiation apparatus and radiating the light through a window of synthetic quartz. However, the window of synthetic quartz is not only an expensive consumable but gives a loss of light.

The outer diameter of the double cylinder lamp is about 25 mm. The difference between the distance from just below the lamp axis to the irradiated surface and the distance from the side of the lamp to the irradiated surface cannot be eliminated from consideration, and a significant difference in illuminance is caused. Therefore, a uniform illuminance distribution cannot be obtained even though the alignment of multiple lamps in close contact with each other. An irradiation apparatus having a window of synthetic quartz can establish a uniform distance and a uniform illuminance distribution in an oxygen atmosphere.

When using the electrodeless field discharge, it is not necessary that the external electrode is made of the mesh electrode. When disposing an external electrode only on a part of the external surface of the lamp, the glow discharge spreads throughout the entire discharge space. The external electrode is typically composed of an aluminum block that also functions as a light reflector and is disposed on the back of the lamp. However since the outer diameter of the lamp is large similarly to that in the dielectric-barrier discharge, the synthetic quartz is required for a uniform illuminance distribution.

The greatest advantage of a fine tube excimer lamp is a simple structure. A gas used for the excimer emission is only sealed inside a quartz tube with the both ends of the tube being closed. The outer diameter of the tube of the fine tube lamp is approximately 6 to 12 mm, and a large diameter requires a high start-up voltage.

The form of discharge can be either dielectric-barrier discharge or electrodeless electric field discharge. A shape of each electrode may have a flat contact surface in contact with the lamp, but if each electrode has a shape corresponding to a curved surface of the lamp, the electrode can firmly secure the lamp and tightly adheres to the lamp to thereby stabilize discharge. In addition, the curved surface composed of an aluminum mirror surface also serves as a light reflector.

Since a Xe excimer lamp radiates an ultraviolet ray having a single wavelength of a short wavelength of 172 nm, the lamp has excellent light emission efficiency. Since the light from such a Xe excimer lamp has a large absorption coefficient to oxygen, radical oxygen atomic species and ozone can be generated in a high concentration by a slight amount of oxygen.

Furthermore, it is known that energy of the light having a short wavelength of 172 nm has high capability of disassociating bonding of an organic substance. The high energy of the active oxygen, ozone, and the ultraviolet rays can realize modification of the polysilazane layer within a short time.

Therefore, in comparison with the low-pressure mercury lamp and the plasma cleaning which emit light having wavelengths of 185 nm and 254 nm, it becomes possible to achieve a reduction in the process time along with high throughput and a reduction in the installation area.

Since the excimer lamp has high generation efficiency of light, the lamp can be driven with a low electric power. Furthermore, since the excimer lamp radiates energy in the ultraviolet region, namely, at a short wavelength, without generating light having a long wavelength which causes elevation of temperature, there is a feature in which the increase in the surface temperature of the target to be irradiated is suppressed. Accordingly, the excimer lamp is suitable for a flexible resin material such as polyethylene terephthalate (PET), which is considered to be easily affected by heating.

Although oxygen is required for the reaction at the time of the ultraviolet ray irradiations, since oxygen absorbs the vacuum ultraviolet rays, it is preferable to perform the irradiation with vacuum ultraviolet rays in a state of an oxygen concentration as low as possible because of easy lowering of the efficiency at an ultraviolet ray irradiation process. Therefore, the oxygen concentration at the time of the vacuum ultraviolet ray irradiation is preferably within the range of 10 to 10000 ppm, more preferably within the range of 50 to 5000 ppm, and further preferably within the range of 1000 to 4500 ppm.

The gas filling the irradiation atmosphere at the time of the vacuum ultraviolet ray irradiation is preferable a dry inert gas, and more preferably a dry nitrogen gas specifically from the viewpoint of cost advantage. The oxygen concentration can be controlled by measuring the flow rates of the oxygen gas and the inert gas fed into the irradiation chamber and by changing the ratio of the flow rates.

Note that, in the above explanation, although the gas barrier layer 12 having the two layers of the first gas barrier layer and the second gas barrier layer has been explained, the gas barrier layer 12 may be configured by either one of the first gas barrier layer or the second gas barrier layer, and further may be configured by three or more layers including other kinds of layer.

Furthermore, although the lamination order of the first gas barrier layer and the second gas barrier layer is not particularly limited, it is preferable that the first gas barrier layer is provided on the substrate 11 side.

In addition, it is also possible to provide a gas barrier layer 12 having a configuration different from the first gas barrier layer and the second gas barrier layer.

[Light-Scattering Layer]

The organic EL element 10 includes a light-scattering layer 13.

Since, in the light-scattering layer 13, the emitted light h from the organic function layer 16 enters through the smooth layer 14, it is preferable that the average refractive index ns of the light-scattering layer 13 is as close as possible to those of the organic function layer 16 and the adjacent smooth layer 14.

The light-scattering layer 13, at the shortest maximum emission wavelength among the maximum emission wavelengths of the emitted light h from the organic function layer 16, preferably has the average refractive index ns of 1.5 or more, particularly within the range of 1.6 or more and less than 2.5. In this case, the light-scattering layer 13 may be formed by using a single material having the average refractive index ns of 1.6 or more and less than 2.5, or by combining two or more compounds to thereby make the average refractive index ns of 1.6 or more and less than 2.5. In such a mixed system, there is used, as the average refractive index ns of the light-scattering layer 13, a calculation refractive index calculated by sum values obtained by multiplying the refractive index which is inherent to each material by the mixing ratio. Moreover, in this case, the refractive index of each material may be less than 1.6 or 2.5 or more, and the average refractive index ns of the mixed layers may satisfy the range of 1.6 or more and less than 2.5.

Here, the “average refractive index ns” means, when the light-scattering layer is formed by a single material, the refractive index of the single material, and when the light-scattering layer is formed by mixed system, the calculation refractive index calculated by sum values obtained by multiplying the refractive index which is inherent to each material by the mixing ratio.

Furthermore, the light-scattering layer 13 is composed of a mixture of a binder having a low refractive index which is a layer medium and the light-scattering particles each having a high refractive index which is contained in the layer medium, has preferably a light-scattering configuration in which the refractive index difference therebetween is utilized.

The light-scattering layer 13 is a layer that enhances the light talking-out efficiency, and is preferably formed on the outermost surface of the gas barrier layer 12 on the first electrode 15 side.

The binder having a low refractive index has a refractive index nb of less than 1.9, particularly preferably less than 1.6.

Here, the refractive index nb of the binder means, when the light-scattering layer is formed by a single material, the refractive index of the single material, and when the light-scattering layer is formed by mixed system, the calculation refractive index calculated by sum values obtained by multiplying the refractive index which is inherent to each material by the mixing ratio.

Furthermore, the light-scattering particle having a high refractive index has a refractive index np of 1.5 or more, preferably 1.8 or more, and particularly preferably 2.0 or more.

Here, the refractive index np of the light-scattering particle means, when the light-scattering layer is formed by a single material, the refractive index of the single material, and when the light-scattering layer is formed by mixed system, the calculation refractive index calculated by sum values obtained by multiplying the refractive index which is inherent to each material by the mixing ratio.

Moreover, in order to enhance the light-scattering property of the light-scattering layer 13, it is considered that the refractive index difference between the light-scattering particles and the binder is increased, that the thickness of the layer is made large, and that the density of the particle is made large. Among them, preferable is the configuration in which the refractive index difference between the light-scattering particles and the binder is increased, because the influence on other properties is small.

The difference |nb−np| of the refractive indexes between the resin material (binder) as the layer medium and the light-scattering particles contained therein is preferably 0.2 or more, particularly preferably 0.3 or more. When the difference |nb−np| of the refractive indexes between the layer medium and the light-scattering particles is 0.03 or more, the scattering effect is generated in the interface between the layer medium and the light-scattering particles. As the difference |nb−np| of the refractive indexes becomes larger, the refraction in the interface becomes larger, and thus the scattering effect is preferably enhanced.

Specifically, since the it is preferable that the average refractive index ns of the light-scattering layer 13 is within the range of 1.6 or more and less than 2.5, it is preferable, for example, that the refractive index nb of the binder is less than 1.6 and the refractive index np of the light-scattering particles is more than 1.8.

Note that the measurement of the refractive index is carried out in an atmosphere of 25° C. by performing irradiation with a light ray having the shortest maximum emission wavelength among the maximum emission wavelengths of the emitted light h from the organic function layer 16, and by using an Abbe refractometer (DR-M2 manufactured by ATAGO Co., Ltd.).

The light-scattering layer 13 is required to have a thickness to some extent in order to ensure an optical path length for generating scattering. On the other hand, it is necessary to limit the thickness so as not to increase an energy loss due to the absorption. Specifically, it is preferable that the thickness is within the range of 0.1 to 5 μm, more preferable within the range of 0.2 to 2 μm.

(Light-Scattering Particle)

As described above, the light-scattering layer 13 can be a layer for diffusing light due to the difference of the refractive indexes between the layer medium and the light-scattering particles. Therefore, it is required that the light-scattering particles to be contained have little influence other layers and can scatter the emitted light h from the organic function layer 16.

Here, the scattering means a state where the haze value (the ratio of the scattering transmittance to total light transmittance) in a case of the light-scattering layer 13 being a single layer is 20% or more, more preferably 25% or more, and particularly preferably 30% or more. When the haze value is 20% or more, it is possible to enhance the light emission efficiency of the organic EL element 10.

The Haze value is a physical value calculated by receiving (i) the influence of the difference in the refractive index of the composition in the layer, and (ii) the influence of the surface shape. Namely, it is possible to measure the haze value obtained by eliminating the influence by the above (ii), by measuring the haze value by suppressing the surface roughness to be lower than a certain level. Specifically, a haze meter (NDH-5000 manufactured by Nippon Denshoku Industries Co., Ltd., etc.) can be used for the measurement.

For example, it is possible to enhance the light-scattering property by adjusting the particle diameter. Specifically, it is preferable to use transparent particles having a particle size larger than the region that generates the Mie scattering in the visible light region. Accordingly, the average particle size is preferably 0.2 μm or more.

On the other hand, when the particle size is large, it is necessary to increase the thickness of the smooth layer 14 provided for smoothing the roughness of the light-scattering layer 13, and it is disadvantageous from the viewpoints of process load and layer absorption. Therefore, the upper limit of the average particle size is preferably less than 1 μm.

Furthermore, a plurality of kinds of particles can be used for the light-scattering layer 13. It is preferable to use together a plurality of the above light-scattering particles, and it is also possible to use together the other particles except the above light-scattering particles. In such a case, the other particles other than the light-scattering particles preferably contain at least one kind of particles each having an average particle size within the range of 100 nm to 3 μm, and do not contain a particle having an average particle size of 3 μm or more. Particularly, it is preferable to contain at least one kind of particles each having an average particle size within the range of 200 nm to 1 μm, and not to contain a particle having an average particle size of 1 μm or more.

The average particle size of these particles can be measured by, for example, using the machine which utilizes the dynamic light-scattering method such as Nanotrac UPA-EX150 manufactured by Nikkiso Co., Ltd., or by image processing of the electron micrographs.

Moreover, the material of the light-scattering particle is not particularly limited and can be appropriately selected depending on the purpose, and may be an organic fine particle or an inorganic fine particle. Among them, an inorganic fine particle having a high refractive index is particularly preferable.

Examples of the organic fine particles include polymethyl methacrylate beads, acryl-styrene copolymer beads, melamine beads, polycarbonate beads, styrene beads, cross-linked polystyrene beads, polyvinyl chloride beads and benzoguanamine-melamine formaldehyde beads, and the like.

Examples of the inorganic fine particles include an inorganic oxide particle composed of at least one oxide of a metal selected from zirconium, titanium, indium, zinc, tin, antimony and the like. Specific examples of the inorganic oxide particles include ZrO₂, TiO₂, BaTiO₃, Al₂O₃, In₂O₃, ZnO, SnO₂, Sb₂O₃, ITO, SiO₂, ZrSiO₄, zeolite, and the like. Among them, TiO₂, BaTiO₃, ZrO₂, ZnO, and SnO₂ are preferable, and TiO₂ is most preferable. In addition, among the TiO₂, the rutile type is more preferable than the anatase type since the weather resistance of the light-scattering layer 13 and the adjacent layers is enhanced due to low catalytic activity, and since furthermore, the refractive index is high.

In addition, in order to incorporate these light-scattering particles into the light-scattering layer 13 having a high refractive index, it is possible to select whether the particles are subjected to surface treatment for the use or are not subjected to surface treatment for the use, from the viewpoint of enhancement of dispersibility and stability when preparing a dispersion described below.

In a case of performing the surface treatment, examples of the specific surface treatment material include a different kind inorganic oxide such as silicon oxide or zirconium oxide, a metal hydroxide such as aluminum hydroxide, an organic acid such as organosiloxane or stearic acid, and the like. These surface treatment materials may be used alone or in combination of two or more kinds. Among them, from the viewpoint of stability of dispersion, the surface treatment material is preferably a different kind inorganic oxide and/or a metal hydroxide, more preferably a metal hydroxide.

When the inorganic oxide particle is subjected to surface coating treatment with the surface treatment material, a coating amount is preferably within the range of 0.01 to 99% by mass. It is possible to sufficiently obtain an enhancement effect of dispersibility and stability by the surface treatment, by setting the coating amount within the above range. Generally, the coating amount is represented by a mass proportion of the surface treatment material to be used on the surface of the particle relative to the mass of the particle.

In addition, the other materials applicable to the light-scattering particle, which can be suitably used, also include, for example, a quantum dot described in WO 2009/014707 A1 or U.S. Pat. No. 6,608,439.

The light-scattering layer 13 is preferably formed at a thickness corresponding to one light-scattering particle so that the light-scattering particles makes contact with or comes close to the interface of the adjacent smooth layer 14. Accordingly, when the total reflection is generated in the smooth layer 14, the evanescent light oozed out to the light-scattering layer 13 can be scattered by the light-scattering particle, and thus the light taking-out efficiency of the organic EL element 10 is enhanced.

The content of the light-scattering particle in the light-scattering layer 13 is preferably, in terms of a volume package ratio, within the range of 1.0 to 70%, more preferably within the range of 5.0 to 50%. Thereby, it is possible to make the coarseness and fineness of the refractive index distribution in the interface between the light-scattering layer 13 and the adjacent smooth layer 14, and to enhance the light taking-out efficiency by the increase in the light-scattering amount.

In a case where, for example, a medium of the layer is a resin material, the formation method of the light-scattering layer 13 includes formation by dispersing the light-scattering particles in a solution containing the resin material serving as the medium to thereby prepare a coating solution, and then performing coating on the substrate 11. In the solution containing the resin material, there is used a solvent which does not dissolve the light-scattering particle.

The light-scattering particles are actually polydispersion particles and are difficult to be regularly arranged, and thus although the particles have locally a diffraction effect, most of the light changes its direction by scattering to thereby enhance the light taking-out efficiency.

(Medium of Layer)

In order to efficiently take the light that is taken into the adjacent smooth layer 14, into the light-scattering layer 13, it is preferable that the difference in the refractive indexes between the medium of the light-scattering layer 13 and the adjacent smooth layer 14 is small. Specifically, the difference in the refractive indexes between the medium of the light-scattering layer 13 and the adjacent smooth layer 14 is preferably 0.1 or less. In addition, it is also preferable that the binder contained in the light-scattering layer 13 and the adjacent smooth layer 14 are formed of the same material.

Furthermore, it is possible, by adjusting the total thickness of the smooth layer 14 and the light-scattering layer 13, to suppress the intrusion of moisture and the wiring failure due to a level difference of edges in pattering, and then to enhance the scattering property. Specifically, the total thickness of the smooth layer 14 and the light-scattering layer 13 is preferably within the range of 100 nm to 5 μm, particularly preferably within the range of 300 nm to 2 μm.

(Resin)

A well-known resins can be used without limitation as the medium of layer (binder) in the light-scattering layer 13, and examples thereof include: resin films such as acrylic acid esters, methacrylic acid esters, polyethylene terephthalate (PET), polybutylene terephthalate, polyethylene naphthalate (PEN), polycarbonate (PC), polyarylate, polyvinyl chloride (PVC), polyethylene (PE), polypropylene (PP), polystyrene (PS), nylon (Ny), aromatic polyamide, polyether ether ketone, polysulfone, polyether sulfone, polyimide, and polyether imide; a heat resistive transparent film which has a silsesquioxane, a polysiloxane, a polysilazane, a polysiloxazane, or the like as a basic skeleton having an organic and inorganic hybrid structure (for example, product name Sila-DEC, manufactured by Chisso Corporation; and a perfluoroalkyl group-containing silane compound (for example, heptadecafluoro-1,1,2,2-tetradecyl)triethoxysilan), as well as a fluorine-containing copolymer having, as a constituent unit, a fluorine-containing monomer and a monomer for imparting a cross-linkable group, and the like. These resins can be used by mixture of two or more kinds. Among them, the resin having the organic inorganic hybrid structure is preferable.

It is also possible to use a hydrophilic resin as the medium of layer. The hydrophilic resins include a water-soluble resin, a water-dispersible resin, a colloidal dispersion resin or a mixture thereof. Examples of the hydrophilic resins include polymers such as an acrylic-based resin, a polyester-based resin, a polyamide-based resin, a polyurethane-based resin and a fluorine-containing resin, and examples include polyvinyl alcohol, gelatin, polyethylene oxide, polyvinyl pyrrolidone, casein, starch, agar, carrageenan, polyacrylic acid, polymethacrylic acid, polyacrylamide, polymethacryl amide, polystyrene sulfonic acid, cellulose, hydroxyl ethyl cellulose, carboxyl methyl cellulose, hydroxyl ethyl cellulose, dextran, dextrin, pullulan and a water-soluble polyvinyl butyral, and among them, polyvinyl alcohol is preferable.

In addition, similarly, a known resin particle (emulsion) and the like can also be suitably used as the medium of layer.

Furthermore, a resin curable mainly by ultraviolet ray or electron beam, namely, a mixed resin in which a thermoplastic resin and a solvent are blended in an ionizing radiation-curable resin, or a thermosetting resin can also be suitably used as the medium of layer. Preferable of such a resin is a polymer having a saturated hydrocarbon or polyether as a main chain, more preferably a polymer having a saturated hydrocarbon as a main chain.

Further, it is preferable that the above medium of layer is cross-linked. A polymer having a saturated hydrocarbon as a main chain is preferably obtained by polymerization of ethylenically unsaturated monomers. In order to obtain a crosslinked binder, it is preferable to use a monomer having two or more ethylenically unsaturated groups.

The resin used as the medium of layer may be used alone, or in combination of two or more as occasion demand.

(Silicone Resin)

There is suitably used, as the medium of layer constituting the light-scattering layer 13, a compound capable of forming a metal oxide, a metal nitride or a metal oxide nitride by ultraviolet ray irradiation under the specified atmosphere. A compound that can be easily subjected to modification at a relatively low temperature described in Japanese Patent Laid-Open No. 08-112879 is preferable as the compound.

Specific examples include a polysiloxane (including polysilsesquioxane) having a Si—O—Si bond, a polysilazane having a Si—N—Si bond, and a polysiloxazane having the both Si—O—Si bond and Si—N—Si bond, and the like. These can be used by mixing two or more kinds. Furthermore, it is possible to use a configuration in which the different compounds are sequentially laminated, or a configuration in which the different compounds are simultaneously laminated.

(Polysiloxane)

The polysiloxane used in the light-scattering layer 13 can include, as the general structure units, [R₃SiO_{1/2}], [R₂SiO], [RSiO_{3/2}] and [SiO₂]. Here, R is independently selected from the group consisting of hydrogen atom, an alkyl group having 1 to 20 carbon atoms (for example, methyl, ethyl, propyl, or the like), an aryl group (for example, phenyl, or the like), and an unsaturated alkyl group (for example, vinyl, or the like).

Examples of the specific polysiloxane groups include [PhSiO_{3/2}], [MeSiO_{3/2}], [HSiO_{3/2}], [MePhSiO], [Ph₂SiO], [PhViSiO], [ViSiO_{3/2}], [MeHSiO], [MeViSiO], [Me₂SiO], [Me₃SiO_{1/2}], and the like. Note that Vi represents a vinyl group.

Mixtures and copolymers of polysiloxane can also be used.

(Polysilsesquioxane)

In the light-scattering layer 13, it is preferable to use the polysilsesquioxane among the above-described polysiloxanes. The polysilsesquioxane is a compound containing a silsesquioxane as a structural unit. The silsesquioxane is a compound represented by [RSiO_{3/2}], and is usually RSiX₃, and the like. R is hydrogen atom, an alkyl group, an alkenyl group, an aryl group, aralkyl group (also, referred to as aralkyl group), and X is a halogen, an alkoxy group, and the like.

There have been known, as a typical shape of the molecular arrangement of the polysilsesquioxane, an amorphous structure, a ladder-like structure, a cage-type structure, and a structure in which one silicon atom is removed from the cage-type structure, a partial cleavage structure in which the silicon-oxygen bond in the cage-type structure is partially cleaved, and the like.

Among these polysilsesquioxanes, it is preferable to use a so-called hydrogen silsesquioxane polymer. Examples of the hydrogen silsesquioxane polymer includes a hydridosiloxane polymer represented by [HSi(OH)ₓ(OR)_{y}O_{z/2}]. Each R is an organic group or a substituted organic group, and when bonded to silicon via the oxygen atom, a hydrolyzable substituent is formed.

Examples of R include an alkyl group (for example, methyl group, ethyl group, propyl group, butyl group, or the like), an aryl group (for example, phenyl group, or the like), an alkenyl group (for example, allyl group, vinyl group, or the like). These resins may be completely condensed) (HSiO_{3/2})ₙ, or only partially hydrolyzed (i.e., including a part of Si—OR), and/or partially condensed (i.e., including a part of Si—OH). In addition, x=0 to 2, y=0 to 2, z=1 to 3, and x+y+z=3.

(Polysilazane)

The polysilazane used in the light-scattering layer 13 is a polymer having a silicon-nitrogen bond, and an inorganic precursor polymer of SiO₂, Si₃N₄ and an intermediate solid solution SiOₓN_{y} (x=0.1 to 1.9, y=0.1 to 1.3) from the both, composed of Si—N, Si—H, N—H, or the like.

The polysilazanes to be preferably used in the light-scattering layer 13 is the polymer represented by the General formula (A) shown in the Chemical formula 1, which is used for the second gas barrier layer constituting the gas barrier layer 12 as described above.

From the viewpoint of denseness of the obtained light-scattering layer 13, the perhydropolysilazane (PHPS) which is a compound in which R¹, R² and R³ in the General formula (A) are all hydrogen atom is particularly preferable.

The polysilazane is commercially available in the form of a solution dissolved in an organic solvent, and the commercially available product can be used as it is as the polysilazane-containing coating solution. Examples of the commercially available polysilazane solutions include NN120-20, NAX120-20, NL120-20, and the like, manufactured by AZ Electronic Materials Co., Ltd.

(Ionizing Radiation-Curable Resin Composition)

An ionizing radiation-curable resin composition can be used as the medium of layer constituting the light-scattering layer 13. The ionizing radiation-curable resin composition can be cured by a usual method for curing the ionizing radiation-curable resin composition, that is, by performing irradiation with an electron beam or an ultraviolet ray.

For example, in case of the electron beam curing, there can be used an electron beam which is emitted from any of various electron beam accelerators of cock Krumlov Walton type, Van de Graaff type, resonance transformer type, insulated core transformer type, linear type, Dynamitron type, high frequency type and the like, and which has an energy within the range of 10 to 1000 keV, preferably within the range of 30 to 300 keV. In a case of the ultraviolet ray curing, there can be used an ultraviolet ray emitted from an ultra-high pressure mercury lamp, a high pressure mercury lamp, a low pressure mercury lamp, a carbon arc, a xenon arc, a metal halide lamp, or the like.

[Smooth Layer]

The smooth layer 14 has a configuration of being mainly composed of the oxide or nitride of Ti having an amorphous structure, or the oxide or nitride of Zr having an amorphous structure. The smooth layer 14 may be formed by a dry process, or may be formed by a wet process, as long as the smooth layer 14 can form the above amorphous structure.

(Wet Process)

In a case where a precursor layer of the smooth layer 14 having the amorphous structure is formed by the wet process, any known methods can be employed. For example, it is preferable to use a compound which contains Ti atom or Zr atom and is applicable to a known sol gel method. These compounds may be obtained by hydrolyzing or polycondensing a tetraalkoxy compound represented by the following General formula (I) and an organoalkoxy compound represented by the following General formula (II). A percentage of the unit of General formula (I) is 50% by volume or more, more preferably 60% by volume or more, further preferably 75% by volume or more.

M¹(OR⁴)(OR⁵)(OR⁶)(OR⁷) (I)

In the General formula (I), M¹ represents an element selected from the group consisting of Ti and Zr, and each of R⁴ to R⁷ represents independently a hydrocarbon group having 1 to 18 carbon atoms.

Further, in the General formula (I), each of R⁴ to R⁷ is more preferably independently a hydrocarbon group having 1 to 8 carbon atoms, particularly preferably a hydrocarbon group having 1 to 5 carbon atoms.

M²(OR⁸)ₐR⁹₄₋ₐ (II)

In the General formula (II), M² represents an element selected from the group consisting of Ti and Zr, and each of R⁸ and R⁹ represents independently hydrogen atom or a hydrocarbon group, and a represents an integer of 2 or 3.

The hydrocarbon group of R⁴ in the above General formula (I) is preferably an alkyl group or an aryl group. The alkyl group preferably has 1 to 18 carbon atoms, more preferably 1 to 8 carbon atoms, further preferably 1 to 4 carbon atoms. Moreover, the aryl group is preferably phenyl group.

Further, the alkyl group or the aryl group may or may not have a substituent. The substituent to be able to be introduced is a halogen atom, amino group, mercapto group, and the like. The above compound represented by the General formula (I) is a low molecular compound, and preferably has a molecular weight of 1000 or less.

The hydrocarbon group of each R⁵ and R⁶ in the above General formula (II) is preferably an alkyl group or an aryl group. The alkyl group preferably has 1 to 18 carbon atoms, more preferably 1 to 8 carbon atoms, further preferably 1 to 4 carbon atoms. Moreover, the aryl group is preferably phenyl group.

Further, the alkyl group or the aryl group may or may not have a substituent. The substituent to be able to be introduced is a halogen atom, an acyloxy group, an alkenyl group, an acryloyloxy group, methacryloyloxy group, amino group, an alkylamino group, mercapto group, epoxy group, and the like.

Each of R⁵ and R⁶ in the General formula (II) is preferably a hydrocarbon group.

Hereinafter, specific examples of the tetraalkoxide compound represented by the General formula (I) will be shown.

Examples of the compounds in which M¹ is Ti include Tetramethoxytitanium, tetraethoxytitanium, tetrapropoxytitanium, tetrabutoxytitanium, tetraisobutoxy titanate, diisopropoxydinormalbutoxy titanate, ditertiallybutoxydiisopropoxytitanate, tetratertiallybutoxy titanate, tetraisooctyltitanate, tetrastearylalcoxytitanate, methoxytriethoxytitanium, ethoxytrimethoxytitanium, methoxytripropoxytitanium, ethoxytripropoxytitanium, propoxytrimethoxytitanium, propoxytriethoxytitanium, dimethoxudieethoxytitanium, and the like. These can be used alone or in combination of two or more kinds.

Exemplary compounds in which M¹ is Zr can include, for example, zirconates corresponding to the compounds exemplified as the above tetraalcoxy titanates.

Next, examples of the organoalcoxy compound represented by the General formula (II) are shown.

Examples in which M² is Ti and a is 2 can include dimethyldimethoxytitanium, diethyldimethoxytitanium, propylmethyldimethoxytitanium, dimethyldiethoxytitanium, diethyldiethoxytitanium, dipropyldiethoxytitanium, γ-chloropropylmethyldiethoxytitanium, γ-chloropropyldimethyldimethoxytitanium, chlorodimethyldiethoxytitanium, (p-chloromethyl)phenylmethyldimethoxytitanium, γ-bromopropylmethyldimethoxytitanium, acetoxymethylmethyldiethoxytitanium, acetoxymethylmethyldimethoxytitanium, acetoxypropylmethyldimethoxytitanium, Benzoyloxypropylmethyldimethoxytitanium, 2-(carbomethoxy)ethylmethyldimethoxytitanium, phenylmethyldimethoxytitanium, phenylmethyldiethoxytitanium, phenylmethyldipropoxytitanium, hydroxymethylmethyldiethoxytitanium, N-(methyldiethoxytitaniumpropyl)-O-polyethyleneoxideurethane, N-(3-methyldiethoxytitaniumpropyl)-4-hydroxybutylamide, N-(3-methyldiethoxytitaniumpropyl)gluconeamide, vinylmethyldimethoxytitanium, vinylmethyldiethoxytitanium, vinylmethyldibutoxytitanium, isopropenylmethyldimethoxytitanium, isopropenylmethyldiethoxytitanium, isopropenylmethyldibutoxytitanium, vinylmethylbis(2-methoxyethoxy)titanium, allylmethyldimethoxytitanium, vinyldecylmethyldimethoxytitanium, vinyloctylmethyldimethoxytitanium, vinylphenylmethyldimethoxytitanium, isopropenylphenylmethyldimethoxytitanium, 2-(meth)acryloxyethylmethyldimethoxytitanium, 2-(meth)acryloxyethylmethyldiethoxytitanium, 3-(meth)acryloxypropylmethyldimethoxytitanium, 3-(meth)acryloxypropylmethyldimethoxytitanium, 3-(meth)-acryloxypropylmethylbis(2-methoxyethoxy)titanium, 3-[2-(allyloxycarbonyl)phenylcarbonyloxy]propylmethyldimethoxytitanium, 3-(vinylphenylamino)propylmethyldimethoxytitanium, 3-(vinylphenylamino)propylmethyldiethoxytitanium, 3-(vinylbenzylamino)propylmethyldiethoxytitanium, 3-(vinylbenzylamino)propylmethyldiethoxytitanium, 3-[2-(N-vinylphenylmethylamino)ethylamino]propylmethyldimethoxytitanium, 3-[2-(N-isopropenylphenylmethylamino)ethylamino]propylmethyldimethoxytitanium, 2-(vinyloxy)ethylmethyldimethoxytitanium, 3-(vinyloxy)propylmethyldimethoxytitanium, 4-(vinyloxy)butylmethyldiethoxytitanium, 2-(isopropenyloxy)ethylmethyldimethoxytitanium, 3-(allyloxy)propylmethyldimethoxytitanium, 10-(allyloxycarbonyl)decylmethyldimethoxytitanium, 3-(isopropenylmethyloxy)propylmethyldimethoxytitanium, 10-(isopropenylmethyloxycarbonyl)decylmethyldimethoxytitanium, 3-[(meth)acryloxypropyl]methyldimethoxytitanium, 3-[(meth)acryloxypropyl]methyldiethoxytitanium, 3-[(meth)acryloxymethyl]methyldimethoxytitanium, 3-[(meth)acryloxymethyl]methyldiethoxytitanium, γ-glycidoxypropylmethyldimethoxytitanium, N-[3-(meth)acryloxy-2-hydroxypropyl]-3-aminopropylmethyldiethoxytitanium, O-[(meth)acryloxyethyl]-N-(methyldiethoxytitaniumpropyl)urethane, γ-glycidoxypropylmethyldiethoxytitanium, β-(3,4-epoxycyclohexyl)ethylmethyldimethoxytitanium, γ-aminopropylmethyldiethoxytitanium, γ-aminopropylmethyldimethoxytitanium, 4-aminobutylmethyldiethoxytitanium, 11-aminoundecylmethyldiethoxytitanium, m-aminophenylmethyldimethoxytitanium, p-aminophenylmethyldimethoxytitanium, 3-aminopropylmethylbis(methoxyethoxyethoxy)titanium, 2-(4-pyridylethyl)methyldiethoxytitanium, 2-(methyldimethoxytitaniumethyl)pyridine, N-(3-methyldimethoxytitaniumpropyl)pyrrole, 3-(m-aminophenoxy)propylmethyldimethoxytitanium, N-(2-aminoethyl)-3-aminopropylmethyldimethoxytitanium, N-(2-aminoethyl)-3-aminopropylmethyldiethoxytitanium, N-(6-aminohexyl)aminomethylmethyldiethoxytitanium, N-(6-aminohexyl)aminopropylmethyldimethoxytitanium, N-(2-aminoethyl)-11-aminoundcylmethyldimethoxytitanium, (aminoethylaminomethyl)phenethylmethyldimethoxytitanium, N-3-[(amino(polypropyleneoxy))]aminopropylmethyldimethoxytitanium, n-butylaminopropylmethyldimethoxytitanium, N-ethylaminoisobutylmethyldimethoxytitanium, N-methylaminopropylmethyldimethoxytitanium, N-phenyl-γ-aminopropylmethyldimethoxytitanium, N-phenyl-γ-aminomethylmethyldiethoxytitanium, (cyclohexylaminomethyl)methyldiethoxytitanium, N-cyclohexylaminopropylmethyldimethoxytitanium, bis(2-hydroxyethyl)-3-aminopropylmethyldiethoxytitanium, diethylaminomethyldiethoxytitanium, diethylaminopropylmethyldimethoxytitanium, dimethylaminopropylmethyldimethoxytitanium, N-3-methyldimethoxytitaniumpropyl-m-phenylenediamine, N,N-bis[3-(methyldimethoxytitanium)propyl]ethylenediamine, bis(methyldiethoxytitaniumpropyl)amine, bis(methyldimethoxytitaniumpropyl)amine, bis[(3-methyldimethoxytitanium)propyl]-ethylenediamine, bis[3-(methyldiethoxytitanium)propyl]urea, bis(methyldimethoxytitaniumpropyl)urea, N-(3-methyldiethoxytitaniumpropyl)-4,5-dihydroimidazole, ureidepropylmethyldiethoxytitanium, ureidepropylmethyldimethoxytitanium, acetoamidepropylmethyldimethoxytitanium, 2-(2-pyridylethyl)thiopropylmethyldimethoxytitanium, 2-(4-pyridylethyl)thiopropylmethyldimethoxytitanium, bis[3-(methyldiethoxytitanium)propyl]disulfide, 3-(methyldiethoxytitanium)propylsuccinic anhydride, γ-mercaptopropylmethyldimethoxytitanium, γ-mercaptopropylmethyldiethoxytitanium, isocyanatopropylmethyldimethoxytitanium, isocyanatopropylmethyldiethoxytitanium, isocyanatoethylmethyldiethoxytitanium, isocyanatomethylmethyldiethoxytitanium, sodium salt of carboxyethylmethyltitaniumdiol, sodium salt triacetic acid of N-(methyldimethoxytitaniumpropyl)ethylenediamine, 3-(methyldihydroxytitanium)-1-propanesulfonic acid, diethylphosphateethylmethyldiethoxytitanium, sodium salt of 3-methyldihydroxytitaniumpropylmethylphosphonate, bis(methyldiethoxytitanium)ethane, bis(methyldimethoxytitanium)ethane, bis(methyldiethoxytitanium)methane, 1,6-bis(methyldiethoxytitanium)hexane, 1,8-bis(methyldiethoxytitanium)octane, p-bis(methyldimethoxytitaniumethyl)benzene, p-bis(methyldimethoxytitaniummethyl)benzene, 3-methoxypropylmethyldimethoxytitanium, 2-[methoxy(polyethyleneoxy)propyl]methyldimethoxytitanium, methoxytriethyleneoxypropylmethyldimethoxytitanium, tris(3-methyldimethoxytitaniumpropyl)isocyanurate, [hydroxy(polyethyleneoxy)propyl]methyldiethoxytitanium, N,N′-bis(hydroxyethyl)-N,N′-bis(methylmethoxytitaniumpropyl)ethylenediamine, bis-[3-(methyldiethoxytitaniumpropyl)-2-hydroxypropoxy]polyethyleneoxide, bis[N,N′-(methyldiethoxytitaniumpropyl)aminocarbonyl]polyethyleneoxide, bis(methyldiethoxytitaniumpropyl)polyethyleneoxide.

Examples in which M² is Ti and a is 3 can include methyltrimethoxytitanium, ethyltrimethoxytitanium, propyltrimethoxytitanium, methyltriethoxytitanium, ethyltriethoxytitanium, propyltriethoxytitanium, γ-chloropropyltriethoxytitanium, γ-chloropropyltrimethoxytitanium, chloromethyltriethoxytitanium, (p-chloromethyl)phenyltrimethoxytitanium, γ-bromopropyltrimethoxytitanium, acetoxymethyltriethoxytitanium, acetoxymethyltrimethoxytitanium, acetoxypropyltrimethoxytitanium, Benzoyloxypropyltrimethoxytitanium, 2-(carbomethoxy)ethyltrimethoxytitanium, phenyltrimethoxytitanium, phenyltriethoxytitanium, phenyltripropoxytitanium, hydroxymethyltriethoxytitanium, N-(triethoxytitaniumpropyl)-O-polyethyleneoxideurethane, N-(3-triethoxytitaniumpropyl)-4-hydroxybutylamide, N-(3-triethoxytitaniumpropyl)gluconeamide, vinyltrimethoxytitanium, vinyltriethoxytitanium, vinyltributoxytitanium, isopropenyltrimethoxytitanium, isopropenyltriethoxytitanium, isopropenyltributoxytitanium, vinyltris(2-methoxyethoxy)titanium, allyltrimethoxytitanium, vinyldecyltrimethoxytitanium, vinyloctyltrimethoxytitanium, vinylphenyltrimethoxytitanium, isopropenylphenyltrimethoxytitanium, 2-(meth)acryloxyethyltrimethoxytitanium, 2-(meth)acryloxyethyltriethoxytitanium, 3-(meth)acryloxypropyltrimethoxytitanium, 3-(meth)acryloxypropyltrimethoxytitanium, 3-(meth)-acryloxypropyltris(2-methoxyethoxy)titanium, 3-[2-(allyloxycarbonyl)phenylcarbonyloxy]propyltrimethoxytitanium, 3-(vinylphenylamino)propyltrimethoxytitanium, 3-(vinylphenylamino)propyltriethoxytitanium, 3-(vinylbenzylamino)propyltriethoxytitanium, 3-(vinylbenzylamino)propyltriethoxytitanium, 3-[2-(N-vinylphenylmethylamino)ethylamino]propyltrimethoxytitanium, 3-[2-(N-isopropenylphenylmethylamino)ethylamino]propyltrimethoxytitanium, 2-(vinyloxy)ethyltrimethoxytitanium, 3-(vinyloxy)propyltrimethoxytitanium, 4-(vinyloxy)butyltriethoxytitanium, 2-(isopropenyloxy)ethyltrimethoxytitanium, 3-(allyloxy)propyltrimethoxytitanium, 10-(allyloxycarbonyl)decyltrimethoxytitanium, 3-(isopropenylmethyloxy)propyltrimethoxytitanium, 10-(isopropenylmethyloxycarbonyl)decyltrimethoxytitanium, 3-[(meth)acryloxypropyl]trimethoxytitanium, 3-[(meth)acryloxypropyl]triethoxytitanium, 3-[(meth)acryloxymethyl]trimethoxytitanium, 3-[(meth)acryloxymethyl]triethoxytitanium, γ-glycidoxypropyltrimethoxytitanium, N-[3-(meth)acryloxy-2-hydroxypropyl]-3-aminopropyltriethoxytitanium, O-[(meth)acryloxyethyl]-N-(triethoxytitanium propyl)urethane, γ-glycidoxypropyltriethoxytitanium, β-(3,4-epoxycyclohexyl)ethyltrimethoxytitanium, γ-aminopropyltriethoxytitanium, γ-aminopropyltrimethoxytitanium, 4-aminobutyltriethoxytitanium, 11-aminoundecyltriethoxytitanium, m-aminophenyltrimethoxytitanium, p-aminophenyltrimethoxytitanium, 3-aminopropyltris(methoxyethoxyethoxy)titanium, 2-(4-pyridylethyl)triethoxytitanium, 2-(trimethoxytitaniumethyl)pyridine, N-(3-trimethoxytitaniumpropyl)pyrrole, 3-(m-aminophenoxy)propyltrimethoxytitanium, N-(2-aminoethyl)-3-aminopropyltrimethoxytitanium, N-(2-aminoethyl)-3-aminopropyltriethoxytitanium, N-(6-aminohexyl)aminomethyltriethoxytitanium, N-(6-aminohexyl)aminopropyltrimethoxytitanium, N-(2-aminoethyl)-11-aminoundcyltrimethoxytitanium, (aminoethylaminomethyl)phenethyltrimethoxytitanium, N-3-[(amino(polypropyleneoxy))]aminopropyltrimethoxytitanium, n-butylaminopropyltrimethoxytitanium, N-ethylaminoisobutyltrimethoxytitanium, N-methylaminopropyltrimethoxytitanium, N-phenyl-γ-aminopropyltrimethoxytitanium, N-phenyl-γ-aminomethyltriethoxytitanium, (cyclohexylaminomethyl)triethoxytitanium, N-cyclohexylaminopropyltrimethoxytitanium, bis(2-hydroxyethyl)-3-aminopropyltriethoxytitanium, diethylaminomethyltriethoxytitanium, diethylaminopropyltrimethoxytitanium, dimethylaminopropyltrimethoxytitanium, N-3-trimethoxytitaniumpropyl-m-phenylenediamine, N,N-bis[3-(trimethoxytitanium)propyl]ethylenediamine, bis(triethoxytitaniumpropyl)amine, bis(trimethoxytitaniumpropyl)amine, bis[(3-trimethoxytitanium)propyl]-ethylenediamine, bis[3-(triethoxytitanium)propyl]urea, bis(trimethoxytitaniumpropyl)urea, N-(3-triethoxytitaniumpropyl)-4,5-dihydroimidazole, ureidepropyltriethoxytitanium, ureidepropyltrimethoxytitanium, acetoamidepropyltrimethoxytitanium, 2-(2-pyridylethyl)thiopropyltrimethoxytitanium, 2-(4-pyridylethyl)thiopropyltrimethoxytitanium, bis[3-(triethoxytitanium)propyl]disulfide, 3-(triethoxytitanium)propylsuccinic anhydride, γ-mercaptopropyltrimethoxytitanium, γ-mercaptopropyltriethoxytitanium, isocyanatopropyltrimethoxytitanium, isocyanatopropyltriethoxytitanium, isocyanatoethyltriethoxytitanium, isocyanatomethyltriethoxytitanium, sodium salt of carboxyethyltitaniumdiol, sodium salt of N-(trimethoxytitaniumpropyl)ethylenediamine, 3-(trihydroxytitanium)-1-propanesulfonic acid, diethylphosphateethyltriethoxytitanium, sodium salt of 3-trihydroxytitaniumpropylmethylphosphonate, bis(triethoxytitanium)ethane, bis(trimethoxytitanium)ethane, bis(triethoxytitanium)methane, 1,6-bis(triethoxytitanium)hexane, 1,8-bis(triethoxytitanium)octane, p-bis(trimethoxytitaniumethyl)benzene, p-bis(trimethoxytitaniummethyl)benzene, 3-methoxypropyltrimethoxytitanium, 2-[methoxy(polyethyleneoxy)propyl]trimethoxytitanium, methoxytriethyleneoxypropyltrimethoxytitanium, tris(3-trimethoxytitaniumpropyl)isocyanurate, [hydroxyl(polyethyleneoxy)propyl]triethoxytitanium, N,N′-bis(hydroxyethyl)-N,N′-bis(trimethoxytitaniumpropyl)ethylenediamine, bis-[3-(triethoxytitaniumpropyl)-2-hydroxypropoxy]polyethyleneoxide, bis[N,N′-(triethoxytitaniumpropyl)aminocarbonyl]polyethyleneoxide, bis(triethoxytitaniumpropyl)polyethyleneoxide.

In a case where M² is Zr, namely, there can be included, as a di-functional or tri-functional organoalkoxy zirconate, for example, an organoalkoxy zirconate that is a compound where Ti is replaced by Zr in the above compound exemplified as the di-functional or tri-functional organoalkoxy titanate.

Commercially available products can be used as the tetraalkoxy compound and the organoalkoxy compound. Furthermore, these compounds can also be obtained by the known reaction, for example, the reaction of the metal with the alcohol.

The tetraalkoxy compound and the organoalkoxy compound may be used alone or in combination two or more kinds.

Furthermore, a chelating agent is preferably used together in order to stabilize the above alkoxide. When the chelating agent is used together, the chelating agent coordinates to the alkoxide to thereby be able to suppress and stabilize the reaction of the alkoxide. A minimum required amount of such a chelating agent is preferably used. The content of the chelating agent is within the range of 0.01 to 33% by mole relative to the unit of the General formula having the alkoxide group, preferably within the range of 0.02 to 15% by mole, more preferably within the range of 0.03 to 5% by mole, and particularly preferably within the range of 0.05 to 1% by mole.

The chelating agent is not particularly limited, and is preferably at least one selected from the group consisting of a β-diketone, a β-ketoester, a polyhydric alcohol, an alkanolamine and an oxycarboxylic acid, from the viewpoint of of enhancing stability against the hydrolysis of the alkoxide compound, and the like.

Examples of the β-diketone compounds include 2,4-pentanedione, 2,4-hexanedione, 2,4-heptanedione, dibenzoylmethane, thenoyltrifluoroacetone, 1,3-cyclohexanedione, 1-phenyl-1,3-butanedione, and the like. Examples of the β-ketoester include methyl acetoacetate, ethyl acetoacetate, propyl acetoacetate, butyl acetoacetate, methyl pivaloylacetate, methyl isobutyloylacetate, methyl caproylacetate, methyllauroylacetate, and the like. Examples of the polyhydric alcohol include 1,2-ethanediol, 1,2-propanediol, 1,2-butanediol, 1,2-pentanediol, 2,3-butanediol, 2,3-pentanediol, glycerin, diethylene glycol, glycerin, hexylene glycol, and the like. Examples of the alkanolamine include N,N-diethylethanolamine, N-(β-aminoethyl)ethanolamine, N-methylethanolamine, N-methyldiethanolamine, N-ethylethanolamine, N-n-butylethanolamine, N-n-butyldiethanolamine, N-tert-butylethanolamine, N-tert-butyldiethanolamine, triethanolamine, diethanolamine, monoethaolamine, and the like. Examples of the oxycarboxylic acids include glycolic acid, lactic acid, tartaric acid, citric acid, malic acid, gluconic acid, and the like. These compounds can be used alone or in combination of two or more kinds.

Furthermore, in the wet process, it is preferable that the alkoxide is subjected to hydroxyl substitution condensation, and then reaction is caused to progress to an oligomer having a certain molecular weight. The molecular weight of the oligomer is, as an average, preferably dimer to 50mers, more preferably trimer to 30mers. These oligomers can be produced by a known method. Particularly, it is preferable that water is supplied to a state where the above chelating agent is coordinated to the alkoxide, and the reaction is caused to gradually progress.

The method for subjecting the alkoxide to hydroxyl substitution condensation is not particularly limited. For example, it is preferable to perform the hydroxyl substitution condensation of the alkoxide by reacting the alkoxide compound or the chelate compound with water in an alcohol solution.

With respect to the amount of water used for oligomerizing by performing condensation, the mole of water is preferably 0.05 to 5.0 moles, more preferably 0.1 to 3.0 moles, and particularly preferably 0.2 to 2.0 moles relative to one mole of the alkoxide compound and/or chelate compound, namely, one mole of the titanium atom or the zirconium atom.

At the time of condensation by hydrolysis, it is preferable to obtain the alkoxide compound oligomer (al) by using a solvent such as an alcohol, and through a heat treatment such as refluxing, as necessary. The alcohol used at this time is not particularly limited, and is preferably an alcohol of the above formula (I) having alkyl groups R⁴ to R⁷ from the viewpoint that the reactivity of the alkoxide compound oligomer is not changed. Specific examples include methanol, ethanol, n-propanol, isopropanol, n-butanol, isobutanol, t-butanol, 2-ethylhexanol, and the like.

The use amount of the alcohol is not particularly limited. It is preferable to carry out dilution by using the alcohol so that the amount of water used for oligomerizing by performing condensation reaches a concentration of 0.5 to 20% by mass in the alcohol solvent, further preferably a concentration of 0.7 to 15% by mass, and particularly preferably a concentration of 1.0 to 10% by mass.

Furthermore, the chelating agent is preferably used together for stabilizing the oligomerized alkoxide compound. A minimum required amount of the chelating agent is preferably used. The content of the chelating agent is within the range of 0.01 to 33% by mole relative to the unit of the General formulae (I) and (II) having the alkoxide group, preferably within the range of 0.02 to 15% by mole, more preferably within the range of 0.03 to 5% by mole, and particularly preferably within the range of 0.05 to 1% by mole.

The oligomerized alkoxide compound preferably has a configuration in which the chelating agent is further coordinated. Namely, a compound in which a chelating agent is further coordinated with the alkoxide compound represented by the General formula (I), or a compound having a configuration in which the chelate compound having a configuration in which the chelating agent is coordinated is condensed with the alkoxide compound is also preferable. That is, the compound having a configuration in which the chelating agent is reacted before and/or after the condensation is preferable from the viewpoint that the stability against the hydrolysis of the alkoxide compound oligomer is enhanced.

The chelating agent used after the condensation is not particularly limited, and the above-described chelating agent can be suitably used. Particularly preferable are the β-diketone, the β-ketoester, and the alkanolamine.

As described above, when the precursor layer of the smooth layer 14 formed by the wet process is subjected to the excimer treatment described below, it is possible to form the smooth layer 14 having an amorphous structure.

(Dry Process)

Next, there will be explained a method for forming a layer serving as a precursor of the smooth layer 14 having an amorphous structure by a dry process.

Any known method can be used as a method for forming a precursor layer of the smooth layer 14 having an amorphous structure by a sputtering method. For example, a substrate is set in a magnetron sputtering apparatus through the use of a target of titanium or zirconium in a vacuum chamber, then titanium is obtained by generation of plasma near the target, and then the titanium is oxidized to apply the precursor layer of the smooth layer 14 on the light-scattering layer 13.

In order to form the precursor layer of the smooth layer 14 having the amorphous structure on the substrate, the upper limit of a sputtering temperature is preferably 80° C., more preferably 50° C., and the lower limit is preferably 0° C., more preferably 5° C. The sputtering temperature is most preferably carried out at an environmental temperature. When carrying out at the environmental temperature, the temperature control is not necessary, and the cost for driving the sputtering machine can be significantly reduced.

With respect to a thickness of thus formed precursor layer of the smooth layer 14, the upper limit is preferably 1000 nm, more preferably 750 nm, and further preferably 500 nm; and the lower limit is preferably 25 nm, more preferably 50 nm, and further preferably 75 nm. When the thickness is less than the lower limit, a sufficient water vapor shielding effect cannot be obtained, and when the thickness is more than the upper limit, the cost is increased.

The formation of the precursor layer of the smooth layer 14 on the light-scattering layer 13 is all preferably carried out by using the sputtering method. It is also preferable to employ the same sputtering conditions. The sputtering temperature is preferably an environmental temperature. Furthermore, a pressure in the vacuum chamber is preferably 0.01 to 3 Pa, more preferably 0.01 to 0.3 Pa. Accordingly, the formation of each layer can be more easily and economically carried out.

The smooth layer 14 having an amorphous structure can be formed by performing the electron beam treatment or the excimer treatment of 150 nm or more and 250 nm or less on thus formed precursor layer of the smooth layer 14 by the dry process.

(Amorphization)

In the smooth layer 14, the amorphous structure was formed by irradiating the precursor layer formed by the above wet process or the dry process with the vacuum ultraviolet ray or the low power electron beam.

In the step of performing irradiation with the vacuum ultraviolet ray, an illuminance of the vacuum ultraviolet ray with which the surface of the precursor layer is to be irradiated is preferably within the range of 30 to 200 mW/cm², more preferably within the range of 50 to 160 mW/cm². When the illuminance is 30 mW/cm² or more, there is no risk of lowering the modification efficiency, and when the illuminance is 200 mW/cm² or less, it is preferable that the ablation is not generated in the precursor layer, and thus no damage is caused to the substrate 11.

An irradiation energy quantity of the vacuum ultraviolet ray on the irradiation surface of the precursor layer is preferably within the range of 200 to 10000 mJ/cm², more preferably within the range of 500 to 5000 mJ/cm². When the energy quantity is 200 mJ/cm² or more, it is possible to sufficiently modify the precursor layer, and when the energy quantity is 10000 mJ/cm² or less, the modification is not excessive, and there is no cracking and no thermal deformation of the substrate 11.

The excimer lamp using a rare gas which is used for forming the above gas barrier layer 12 is preferably used as the vacuum ultraviolet ray source.

Although oxygen is necessary in the reaction at the time of ultraviolet ray irradiation, the reaction efficiency in the process of ultraviolet ray irradiation is easily lowered because the vacuum ultraviolet ray is absorbed by oxygen. Therefore, it is preferable that vacuum ultraviolet ray irradiation is performed in a state of an oxygen content being as low as possible. Namely, the oxygen content at the time of the vacuum ultraviolet ray irradiation is preferably within the range of 10 to 10000 ppm, more preferably within the range of 50 to 5000 ppm, and further preferably within the range of 1000 to 4500 ppm.

(Amorphous Structure)

According to the above producing method, the smooth layer 14 has the amorphous structure containing the Ti atom or the Zr atom.

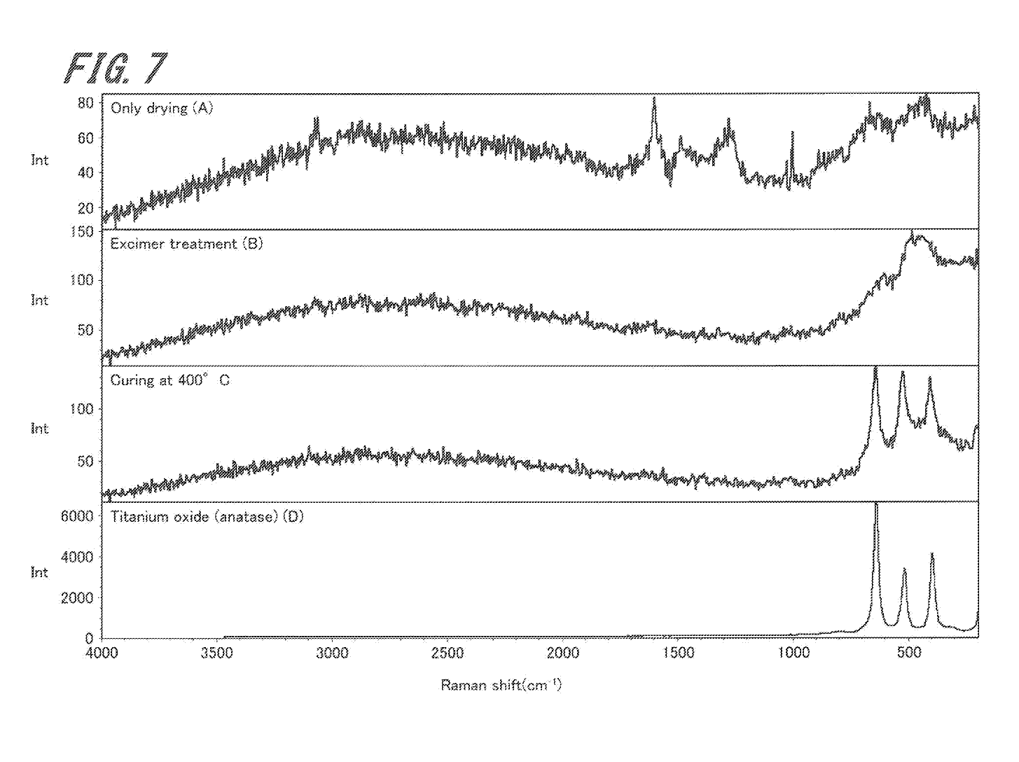

The amorphous structure can be detected by the spectrum peak defined by the Raman spectroscopic absorption, the X ray analysis, or the like. In the following, the amorphous structure of the smooth layer 14 is explained by referring the Raman spectroscopic absorption spectrum of the TiO₂ shown in FIG. 7. In FIG. 7, the TiO₂ Raman spectroscopic absorption spectrums of the different states from each other are shown in (A) to (D), respectively. The (D) shows the Raman spectroscopic absorption spectrum of the (anatase type) titanium crystal.

As shown in (A) of FIG. 7, in the formation of TiO₂, only when the above alkoxide-containing sol gel solution is applied and dried, the peak derived from the (anatase type) titanium crystal cannot be observed, and thus, the crystallization of the Ti is no proceeded yet. At this time, the refraction index is still low. Further, since the reaction is not completed, the peaks derived from unreacted organic substances are observed in 1000 to 2000 cm⁻¹. In addition, at this time, it has been also confirmed that the outgas (for example, gas released at 190° C. for 30 minutes) which gives an adverse effect to the organic EL element 10 is released in a large amount from the layer.

On the other hand, when treating at a high temperature of a known 300° C. or more, as shown in (C) of FIG. 7, the spectrum of the Raman spectrum 300 to 700 cm⁻¹ is changed, the peak derived from the (anatase type) titanium crystal can be clearly observed. Note that, (C) of FIG. 7 shows the spectrum distribution of the sample treated at 400° C. for 60 minutes.

In addition, the layer of TiO₂ is shrunk to be highly dense by the above crystallization, the refractive index is improved remarkably, and the above outgas is not released. However, the TiO₂ layer becomes a layer having photocatalytic activity. In the formation of the TiO₂ layer by using the sol gel reaction, since the layer becomes photocatalytic, there is a problem that the long pot life and the weather resistance in outdoor are considerably lowered.

Further, since the crystallized TiO₂ layer does not have the flexibility adequacy, it is not suitable to the organic EL element to which the flexibility adequacy is required.

In contrast, in the smooth layer as shown in (B) of FIG. 7, with respect to the TiO₂ layer having the amorphous structure, the peak derived from the (anatase type) titanium crystal cannot be observed, and also the peak in 1000 to 2000 cm⁻¹ is not observed, and the refractive index is extremely improved and the outgas is not released. Note that, (B) of FIG. 7 shows the spectrum distribution of the sample where the amorphous structure is formed by the above excimer treatment.

When the uniform amorphous structure is formed by irradiating the light from the above high power excimer lamp of the deep UV light, or the low power electron beam, the refractive index is improved as same as the crystallized TiO₂ layer, and the outgas is also considerably reduced. Further, different from the crystallized TiO₂ layer, the TiO₂ layer having the amorphous structure has good long pot life and weather resistance in outdoor.

In addition, when having the amorphous structure, it is possible to endow the TiO₂ layer with the flexibility adequacy. With respect to the organic EL element to which the flexibility adequacy is required, the smooth layer 14 having the amorphous structure containing Ti atom or Zr atom can be applied.

The same effects are confirmed by applying the Xe excimer light source of 172 nm, the KrCl excimer light source of 222 nm, and EB engine of the electron beam irradiation machine manufactured by Hamamatsu Photonics Co., Ltd. The same effects are confirmed under the atmospheric circumstance or under a circumstance where the oxygen content is lowered, even though there is a difference in the effects.

[First Electrode, Second Electrode]

The organic EL element 10 is provided with the first electrode 15 and the second electrode 17 as a pair of the electrodes for sandwiching the organic function layer 16. In the organic EL element 10, one of the first electrode 15 and the second electrode 17 functions as an anode, and the other functions as a cathode.

Furthermore, the first electrode 15 which is provided on the light taking-out side of the substrate 11 is preferably a transparent electrode. Note that the transparency means that the light transmittance at a wavelength of 550 nm is 50% or more.

Hereinafter, explanation will be made of the configuration in which the first electrode 15 is an anode formed by a transparent electrode, and the second electrode 17 is a cathode serving as a reflective electrode. Note that, in the organic EL element 10, it is possible to arbitrarily combine the first electrode 15 and second electrode 17, and the anode and cathode, for application.

(First Electrode)

The first electrode 15 is preferably constituted by silver or an alloy mainly composed of silver.

Here, the main component means a component which has the highest percentage among the components composing the first electrode 15.

Examples of the alloys constituting the first electrode 15 and being mainly composed of silver (Ag) include silver magnesium (AgMg), silver copper (AgCu), silver palladium (AgPd), silver palladium copper (AgPdCu), silver indium (AgIn), and the like.

The first electrode 15 as described above may have a configuration in which layers of silver or layers of the alloy mainly composed of silver are laminated dividedly into a plurality of layers, as necessary.

In order to efficiently taking out emitted light from the first electrode 15, it is desirable that the transmittance is increased more than 10%, and a sheet resistance of the anode is preferably hundreds of Ω/square or less.

The thickness of the first electrode 15 is preferably within the range of 2 to 15 nm, more preferably within the range of 3 to 12 nm, and particularly preferably within the range of 4 to 9 nm. When the thickness is less than 15 nm, the absorption components or the reflection components of the layer are small, and thus the transmittance of the transparent electrode becomes large. Furthermore, when the thickness is more than 2 nm, it is possible to ensure the conductivity of the layer.

Moreover, when the first electrode 15 is used as the anode, an electrode material composed of a metal having a high work function (4 eV or more), an alloy, an electrically conductive compound, or a mixture thereof are preferably used. Examples of such electrode materials include a metal such as Au, an electrically conductive transparent material such as CuI, indium tin oxide (ITO), SnO₂ or ZnO. In addition, a material capable of producing an amorphous transparent conductive film such as IDIXO (In₂O₃—ZnO) may also be used.

In the first electrode 15, thin films of these electrode materials may be formed by a method such as vapor deposition or sputtering, and a pattern having a desired shape may be formed by photolithography. Furthermore, in a case where high patterning accuracy is not necessary so much (approximately 100 μm or more), a certain pattern may be formed via a mask having a desired shape at the time of vapor deposition or sputtering of the electrode material.

Alternatively, in a case where a material capable of being coated such as an electrically conductive organic compound is used, a wet film forming method such as a printing method or a coating method can also be used.

(Second Electrode)

The second electrode 17 as described above is, as similar to the above first electrode 15, preferably constituted using silver or an alloy mainly composed of silver.

Here, the main component means a component having the highest percentage among the components composing the second electrode 17.

Examples of the alloys constituting the second electrode 17 and being mainly composed of silver (Ag) include silver magnesium (AgMg), silver copper (AgCu), silver palladium (AgPd), silver palladium copper (AgPdCu), silver indium (AgIn), and the like.

The second electrode 17 as described above may have a configuration in which layers of silver or layers of the alloy mainly composed of silver are laminated dividedly into a plurality of layers, as necessary.

Furthermore, when the second electrode 17 is used as the cathode, an electrode material composed of a metal having a low work function (4 eV or less) (referred to as an electron-injecting metal), an alloy, an electrically conductive compound, or a mixture thereof are preferably used.

Specific examples of such electrode materials as described above include sodium, a sodium-potassium alloy, magnesium, lithium, a magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, a rare earth metal, and the like.

Among them, from the viewpoint of electron injection property and durability against oxidation, preferred examples are a mixture of the electron-injecting metal and a secondary metal that is a metal having a work function higher than that of the electron-injecting metal and being stable, such as a magnesium/silver mixture, a magnesium/aluminum mixture, a magnesium/indium mixture, an aluminum/aluminum oxide (Al₂O₃) mixture, a lithium/aluminum mixture, aluminum, and the like.

The second electrode 17 can be produced by forming a thin film of the electrode material by a method such as vapor deposition or sputtering.

The sheet resistance of the second electrode 17 is preferably several hundred Q/square or less, and the thickness thereof is usually selected within the range of 10 nm to 5 μm, preferably within the range of 50 nm to 200 nm.

Furthermore, when the second electrode 17 is produced at a thickness of 1 to 20 nm, the electrically conductive transparent materials mentioned in the explanation of the first electrode 15 is produced thereon, and thus a transparent or translucent second electrode 17 can be produced, and it is possible to produce an element in which both of the first electrode 15 and the second electrode 17 have a light-transmitting property by utilizing this technique.

Note that, in a case where the emitted light h is also taken out from the second electrode 17 in the organic EL element 10, the second electrode 17 may be constituted by selection of a conductive material having a good light-transmitting property from the above-described conductive materials. The organic EL element 10 can have a both-side light emission type configuration by making the second electrode 17 transparent.

(Base Layer)

In a case where the first electrode 15 and the second electrode 17 are formed by the material mainly composed of silver, the first electrode 15 and the second electrode 17 are preferably formed on the base layer which improves the film-deposition property of a silver thin film.

The base layer is formed adjacent to the first electrode 15 and the second electrode 17, and after the process of forming the base layer, the first electrode 15 and the second electrode 17 are formed. The material for forming the base layer is not particularly limited, and may be a material which can suppress the aggregation of silver at time of deposition of the material mainly composed of silver. Examples of the materials constituting the base layer include a compound containing a nitrogen atom or a sulfur atom, and the like.

In a case where the base layer is composed of a material having a low refractive index (refractive index of less than 1.7), the upper limit of the layer thickness is required to be less than 50 nm, preferably less than 30 nm, further preferably less than 10 nm, and particularly preferably less than 5 nm. When the layer thickness is less than 50 nm, the optical loss can be minimized. On the other hand, the lower limit of the layer thickness must be 0.05 nm or more, preferably 0.1 nm or more, and particularly preferably 0.3 nm or more. When the layer thickness is 0.05 nm or more, the base layer is easily and uniformly formed and the aggregation of silver can be uniformly suppressed.

In a case where the base layer is composed of a material having a high refractive index (refractive index of 1.7 or more), the upper limit is not particularly limited, and the lower limit of the layer thickness is similar to a case of being composed of the above material having a low refractive index.

However, it is sufficient that the base layer may be formed at a necessary layer thickness with which uniform film formation is obtained.

The nitrogen-containing compound composing the base layer is not particularly limited as long as the compound contains a nitrogen atom in the molecule, and is preferably a compound having a heterocyclic ring containing a nitrogen atom as the hetero atom. Examples of the heterocyclic ring containing a nitrogen atom as the hetero atom include aziridine, azirine, azetidine, azete, azolidine, azoles, ajinan, pyridine, azepane, azepine, imidazole, pyrazole, oxazole, thiazole, imidazoline, pyrazine, morpholine, thiazine, indole, isoindole, benzimidazole, purine, quinoline, isoquinoline, quinoxaline, cinnoline, pteridine, acridine, carbazole, benzo-C-cinnoline, porphyrins, chlorins, choline, and the like.

The method for forming the base layer include: a wet process such as an application method, an inkjet method, a coating method or a dipping method; or a dry process such as a vapor deposition method (resistance heating, EB method, and the like), a sputtering method, a CVD method. Among them, the vapor deposition method is preferably applied.

As explained above, the silver atom interacts with the nitrogen-containing compound constituting the base layer by provision of a layer mainly composed of silver, on the base layer constituted using the compound containing a nitrogen atom, and then the diffusion length on the surface of the base layer of the silver atom is decreased and the aggregation of the silver is suppressed.

Generally, in the formation of the layer mainly composed of silver, since the thin film growth is carried out as the result of the growth by a nuclear growth type (Volumer-Weber: VW type), the silver particle tends to be easily isolated in an island shape and when the layer thickness is small, it is difficult to obtain electric conductivity to thereby increase a sheet resistance value. Therefore, although the layer thickness is required to be increased for ensuring the conductivity, the light-transmitting property is lowered when the layer thickness is increased, with the result that the base layer was inappropriate as a transparent electrode.

However, in a case where the layer mainly composed of silver is formed on the above-described base layer, the thin film growth is carried out as the result of the growth of a single layer growth type (Frank-van der Merwe: FM type) due to the interaction between silver and a compound containing a nitrogen atom or sulfur atom, and thus aggregation of the silver is suppressed.

[Organic Function Layer]

The organic EL element 10 has a configuration in which the luminescent organic function layer 16 is provided between the electrodes. The organic function layer 16 has at least the light-emitting layer, and in addition, other layers may be disposed between the light-emitting layer and each of the electrodes.

(Configuration of Organic Function Layer)

A representative elemental configuration of the organic function layer 16 can include the following configuration, but is not limited thereto.
(1) Light-emitting layer
(2) Light-emitting layer/Electron transport layer
(3) Positive hole transport layer/Light-emitting layer
(4) Positive hole transport layer/Light-emitting layer/Electron transport layer
(5) Positive hole transport layer/Light-emitting layer/Electron transport layer/Electron-injecting layer
(6) Positive hole-injecting layer/Positive hole transport layer/Light-emitting layer/Electron transport layer
(7) Positive hole-injecting layer/Positive hole transport layer/(Electron-blocking layer/) Light-emitting layer/(Positive hole-blocking layer/) Electron transport layer/Electron-injecting layer

Among them, the configuration of (7) is preferably used, but is not limited thereto.

In the above configuration, the light-emitting layer is composed of a mono-layer or multi-layer. In a case of the plurality of light-emitting layer, a non-light-emitting intermediate layer may be disposed between the respective light-emitting layers.

In addition, as necessary, a positive hole-blocking layer (positive hole barrier layer), an electron-injecting layer (cathode buffer layer) or the like may be disposed between the light-emitting layer and the cathode. Additionally, an electron-blocking layer (electron barrier layer), a positive hole-injecting layer (anode buffer layer) or the like may be disposed between the light-emitting layer and the anode.

The electron transport layer is a layer having a function of transporting an electron. The electron transport layer also includes the electron-injecting layer, and the positive hole-blocking layer in a broad sense. Furthermore, the electron transport layer may be composed of a plurality of layers.

The positive hole transport layer is a layer having a function of transporting a positive hole. The positive hole transport layer also includes the positive hole-injecting layer, and the electron-blocking layer in a broad sense. Moreover, the positive hole transport layer may be composed of a plurality of layers.

(Tandem Structure)

Furthermore, the organic EL element 10 may be an element of so-called Tandem structure in which a plurality of organic function layers 16 including at least one light-emitting layer is laminated.

A representative example of an element configuration having a tandem structure is as follows.

(1) Anode/first organic function layer/intermediate connector layer/second organic function layer/cathode
(2) Anode/first organic function layer/intermediate connector layer/second organic function layer/intermediate connector layer/third organic function layer/cathode

Here, the above first organic function layer, second organic function layer, and third organic function layer may be all the same or different. Moreover, it may be possible that two organic function layers are the same and the remaining one is different.

In addition, each organic function layer may be directly laminated or may be laminated via the intermediate connector layer. For example, the intermediate connector layer is constituted of an intermediate electrode, an intermediate conductive layer, a charge-generating layer, an electron extraction layer, a connecting layer, an intermediate insulation layer, or the like, and may be made by known material formulation as long as the layer has a function of supplying an electron to an adjacent layer to the anode, and supplying a positive hole to an adjacent layer on the cathode side.

Examples of materials used in the intermediate connector layer include an electrically conductive inorganic compound layer such as ITO (indium tin oxide), IZO (indium zinc oxide), ZnO₂, TiN, ZrN, HfN, TiOₓ, VOₓ, CuI, InN, GaN, CuAlO₂, CuGaO₂, SrCu₂O₂, LaB₆, RuO₂, or Al, a two-layered film such as Au/Bi₂O₃, a multi-layered film such as SnO₂/Ag/SnO₂, ZnO/Ag/ZnO, Bi₂O₃/Au/Bi₂O₃, TiO₂/TiN/TiO₂, or TiO₂/ZrN/TiO₂, a fullerene such as C60, an electrically conductive organic layer such as oligothiophene, and an electrically conductive organic compound layer such as metal phthalocyanine, metal-free phthalocyanine, metal porphyrin, or metal-free porphyrin, and the like, but are not limited thereto.

Examples of the tandem type organic EL element include elemental configurations and materials described in U.S. Pat. No. 6,337,492, U.S. Pat. No. 7,420,203, U.S. Pat. No. 7,473,923, U.S. Pat. No. 6,872,472, U.S. Pat. No. 6,107,734, U.S. Pat. No. 6,337,492, WO 2005/009087, Japanese Patent Laid-Open No. 2006-228712, Japanese Patent Laid-Open No. 2006-24791, Japanese Patent Laid-Open No. 2006-49393, Japanese Patent Laid-Open No. 2006-49394, Japanese Patent Laid-Open No. 2006-49396, Japanese Patent Laid-Open No. 2011-96679, Japanese Patent Laid-Open No. 2005-340187, JP Patent No. 4711424, JP Patent No. 3496681, JP Patent No. 3884564, JP Patent No. 4213169, Japanese Patent Laid-Open No. 2010-192719, Japanese Patent Laid-Open No. 2009-076929, Japanese Patent Laid-Open No. 2008-078414, Japanese Patent Laid-Open No. 2007-059848, Japanese Patent Laid-Open No. 2003-272860, Japanese Patent Laid-Open No. 2003-045676, WO 2005/094130, and the like, but are not limited thereto.

(Positive Hole-Injecting Layer)

In the organic EL element 10, the positive hole-injecting layer (anode buffer layer) may be disposed between the first electrode 15 and the light-emitting layer, or between the first electrode 15 and the positive hole transport layer. Note that the positive hole-injecting layer is disposed between the first electrode 15 and the light-emitting layer or the positive hole transport layer in order to reduce a driving voltage and to improve an emission luminance of the organic EL element 10. The compounds described in Japanese Patent Laid-open No. 2000-160328 can be used as materials for forming the positive hole-injecting layer (anode buffer layer).

(Positive Hole Transport Layer)

The positive hole transport layer is a layer where a positive hole supplied from the first electrode 15 is transported (injected) to the light-emitting layer. In addition, the positive hole transport layer also acts as a barrier that blocks the inflow of electron from the second electrode 17 side. Therefore, the term of “positive hole transport layer” is used in a broad sense, that is, in a sense of including the positive hole-injecting layer and/or the electron-blocking layer.

Any organic or inorganic materials can be used as the positive hole transport material as long as the material can express the function of transporting (injecting) the above positive hole and the function of blocking the inflow of the electron. Specific examples of the positive hole transport materials include compounds such as a triazole derivative, an oxadiazole derivative, an imidazole derivative, a polyarylalkane derivative, a pyrazoline derivative and a pyrazolone derivative, a phenylenediamine derivative, an arylamine derivative, an amino-substituted chalcone derivative, an oxazole derivative, a styrylanthracene derivative, a fluorenone derivative, a hydrazone derivative, a stilbene derivative, a silazane derivative, an aniline-based copolymer, and a conductive high molecular oligomer (particularly a thiophene oligomer).

Furthermore, there can be used, as the positive hole transport material, compounds such as a porphyrin compound, and an aromatic tertiary amine compound (a styrylamine compound). Particularly, according to the present embodiment, it is preferable to use the aromatic tertiary amine compound.

Examples of the aromatic tertiary amine compounds that can be used include N, N,N′,N′-tetraphenyl-4,4′-diaminophenyl, N,N′-diphenyl-N,N′-bis(3-methylphenyl)-[1,1′-biphenyl]-4,4′-diamine (TPD), 2,2-bis(4-di-p-tolylaminophenyl)propane, 1,1-bis(4-di-p-tolylaminophenyl)cyclohexane, N,N,N′,N′-tetra-p-tolyl-4,4′-diaminobiphenyl, 1,1-bis(4-di-p-tolylaminophenyl)-4-phenylcyclohexane, bis(4-dimethylamino-2-methylphenyl)phenylmethane, bis(4-di-p-tolylaminophenyl)phenylmethane, N,N′-diphenyl-N,N′-di(4-methoxyphenyl)-4,4′-diaminobiphenyl, N,N,N′,N′-tetraphenyl-4,4′-diaminodiphenylether, 4,4′-bis(diphenylamino)quadriphenyl, N,N,N-tri(p-tolyl)amine, and the like. Moreover, examples of the aromatic tertiary amine compound include the styrylamine compound such as 4-(di-p-tolylamino)-4′-[4-(di-p-tolylamino)styryl]stilbene, 4-N,N-diphenylamino-(2-diphenylvinyl)benzene, 3-methoxy-4′-N,N-diphenylaminostilbene. Furthermore, those having two condensed aromatic rings in a molecule as described in U.S. Pat. No. 5,061,569, for example, 4,4′-bis[N-(1-naphthyl)-N-phenylamino]biphenyl (NPD), and 4,4′,4″-tris[N-(3-methylphenyl)-N-phenylamino]triphenylamine (MTDATA) in which three triphenylamine units are bonded in a star burst as described in Japanese Patent Laid-Open No. 04-308688 may be used.

Moreover, there can also be used, as the positive hole transport material, for example, polymer materials in which the above various positive hole transport material are introduced into a polymer chain, or polymer materials using the above various positive hole transport materials as a main chain of a polymer. Note that inorganic compounds such as a p-type Si and a p-type SiC can also be used as the positive hole transport material and a formation material of the positive hole-injecting layer.

Furthermore, it may also be possible to use, as the positive hole transport material, so-called p-type positive hole transport materials described in documents such as Japanese Patent Laid-Open No. 11-251067 and the document by J. Huang et. al. (Applied Physics Letters 80 (2002), p. 139). Note that it is possible to obtain a light-emitting element having higher efficiency when using these materials for the positive hole transport material.

Furthermore, according to the present embodiment, the positive hole transport layer having a high p property (positive hole rich) may be formed by doping of the positive hole transport layer with impurities. Examples include those described in documents such as Japanese Patent Laid-Open Nos. 04-297076, 2000-196140, 2001-102175 and J. Appl. Phys., 95, 5773 (2004), and the like. It is possible to produce the organic EL element 10 which consumes lower electric power in the case of using the positive hole transport layer rich in positive hole.

(Light-Emitting Layer)

The light-emitting layer is a layer where the positive hole injected directly from the first electrode 15 or injected via the positive hole transport layer or the like from the first electrode 15, and where the electron injected directly from the second electrode 17 or injected through the electron transport layer or the like from the second electrode 17 are recombined to emit light. Note that the light-emitting part may be inside the light-emitting layer, or may be on the interface between the light-emitting layer and the layer adjacent thereto.

In addition, the light-emitting layer may be a mono-layer or multi-layer. Note that, when a plurality of light-emitting layer is disposed, the configuration may be such that the plurality of the light-emitting layers having different emitting lights is laminated. Furthermore, when a plurality of light-emitting layer is disposed, a non-light-emitting intermediate layer may be disposed between the adjacent light-emitting layers. In this case, the intermediate layer can be formed by the same material as the host compound described below in the light-emitting layer.

According to the present embodiment, the light-emitting layer is formed by an organic light-emitting material which contains a host compound (light-emitting host) and a light-emitting material (light-emitting dopant). In the light-emitting layer having such a configuration, it is possible to obtain any emitting color light by appropriately controlling the luminescent wavelength of the light-emitting material, the kind of the light-emitting material to be included, or the like.

(1. Host Compound)

It is preferable to use, as the host compound contained in the light-emitting layer, a compound having, in phosphorescence emission at room temperature (25° C.), a phosphorescence quantum yield of less than about 0.1. Particularly, a compound having a phosphorescence quantum yield of about less than 0.01 is preferably used. Furthermore, among the various compounds contained in the light-emitting layer, a volume ratio of the host compound in the light-emitting layer is preferably a value of about 50% or more.

Moreover, a known host compound can be used as the host compound. In such a case, the host compound may be used alone or in combination of plural kinds of host compounds. It is possible to adjust a transfer degree (transfer amount) of charges (positive hole and/or electron), and to enhance a light emission efficiency of the organic EL element 10, by using a plurality of host compounds.

There can be used, as the host compound having the above-described properties, a compound such as a well-known low-molecular-weight compound, a high-molecular-weight compound having a repeating unit, or a low-molecular-weight compound having a polymerizable group such as vinyl group or epoxy group (vapor-deposition polymerizable emission host). Note that it is preferable to use, as the host compound, a compound having a positive hole transport function, an electron transport function, a function of preventing the increase in a light emission wavelength, and a high Tg (glass transition temperature). However, the glass transition temperature Tg here is a value obtained by using DSC (Differential Scanning Colorimetry) in accordance with JIS-K 7121.

Specific examples of the host compounds include compounds described in the following documents such as Japanese Patent Laid-Open Nos. 2001-257076, 2002-308855, 2001-313179, 2002-319491, 2001-357977, 2002-334786, 2002-8860, 2002-334787, 2002-15871, 2002-334788, 2002-43056, 2002-334789, 2002-75645, 2002-338579, 2002-105445, 2002-343568, 2002-141173, 2002-352957, 2002-203683, 2002-363227, 2002-231453, 2003-3165, 2002-234888, 2003-27048, 2002-255934, 2002-260861, 2002-280183, 2002-299060, 2002-302516, 2002-305083, 2002-305084 and 2002-308837.

Note that, in the present embodiment, the host compound is preferably a carbazole derivative, particularly preferably a compound of a carbazole derivative and dibenzofuran compound.

(2. Light-Emitting Material)

It is possible to use, as the light-emitting material (light-emitting dopant), for example, a phosphorescence emitting material (a phosphorescent compound, a phosphorescence emitting compound), a fluorescent emitting material, and the like. However, it is preferable to use, as the light-emitting material, the phosphorescence emitting material from the viewpoint of improvement of light emission efficiency.

The phosphorescence emitting material is a compound in which light emission can be obtained from an excited triplet state. Specifically, the phosphorescence emitting material is a compound which emits phosphorescence at room temperature (25° C.) and a phosphorescence quantum yield at 25° C. is about 0.01 or more. However, in the present embodiment, it is preferable to use a phosphorescence emitting material having a value of a phosphorescence quantum yield of about 0.1 or more. Note that the phosphorescence quantum yield can be measured by, for example, a method described on page 398 of “Spectroscopy II of Lecture of Experimental Chemistry vol. 7, 4th edition” (1992, published by Maruzen Co., Ltd.). The phosphorescence quantum yield in a solution can be measured by using various solvents, and in the present embodiment, a light-emitting material in which a phosphorescence emitting material can achieve the above-described phosphorescence quantum yield of about 0.01 or more with one of appropriate solvents may be used.

Furthermore, the light-emitting layer may contain one kind of the light-emitting material, or may contain a plural kinds of light-emitting materials which have different maximum light emission wavelength. When using a plural kinds of light-emitting materials, a plurality of lights each having different wavelengths can be mixed to thereby be able to give light having an optional luminous color. For example, a white color light can be obtained by adding a blue dopant, a green dopant and a red dopant (three kinds of light-emitting materials) to the light-emitting layer.

The following two kinds of processes are included regarding process (principle) of light emission (phosphorescent emission) in the light-emitting layer which contains the host compound and the phosphorescent emitting material described above.

The first light emission process is a process of an energy transfer type. In the light emission process of the type, first, carriers (positive hole and electron) recombine on the host compound in the light-emitting layer where the carriers are transferred to produce an excited state of the host compound. Then, the energy generated at that time is transferred from the host compound to the phosphorescence-emitting material (the energy level of the excited state is transferred from the excited level of the host compound to the excited level (excited triplet)), and as the result, light is emitted from the phosphorescence emitting material.

The second light emission process is a light emission process of a carrier trap type. In the light emission process of the type, the phosphorescence emitting material traps the carriers (positive hole and electron) in the light-emitting layer. As a result, the carriers recombine on the phosphorescence emitting material, and then light is emitted from the phosphorescence-emitting material. In every light emission process described above, it is necessary that the energy level of the excited state of the phosphorescence emitting material is made lower than the energy level of the excited state of the light-emitting host.

The desired phosphorescence-emitting material can be suitably selected, as the phosphorescence emitting material which generates the above-described light emission processes, from among the well-known various phosphorescence-emitting materials (phosphorescence-emitting compounds) used for the conventional organic EL element. For example, a complex-based compound containing a metal of the groups 8 to 10 in the element periodic table can be used as the phosphorescence-emitting material. Even among the complex-based compounds, it is preferable to use any of an iridium compound, an osmium compound, a platinum compound (a platinum complex-based compound) or a rare earth metal complex, as the phosphorescence-emitting material. In the present embodiment, particularly preferable phosphorescence-emitting material to be used is an iridium compound.

In addition, examples of the fluorescence-emitting materials (fluorescence-emitting body, fluorescent dopant) that can be used include a coumarin-based dye, a pyran-based dye, a cyanine-based dye, a croconium-based c dye, a squarylium-based dye, an oxobenzanthracene-based dye, a fluorescein-based dye, a rhodamine-based dye, a pyrylium-based dye, a perylene-based dye, a stilbene-based c dye, a polythiophene-based dye, or a rare earth metal complex-based fluorescent material, and the like.

Note that a color of light emitted by the organic EL element 10 is a color obtained by measuring a color emitted from the organic EL element 10 by a spectroradiometric luminance meter (CS-2000 manufactured by Konica Minolta Sensing, Inc.), and the measured result is applied to the CIE (Commission Internationale de l'Eclairage) chromaticity coordinate (for example, in FIG. 4 0.16 on page 108 of “Shinpen Shikisai Kagaku Handbook” (edited by The Color Science Association of Japan, Tokyo Daigaku Shuppan Kai, 1985)). Specifically, the “white color” used herein means a color which exhibits chromaticity in the CIE 1931 Color Specification System at 1000 cd/m² within the region of X=0.33±0.07 and Y=0.33±0.07, when measurement is done to 2-degree viewing angle front luminance via the aforesaid method.

In addition, the method for obtaining the white color emission is not limited to the method in which a plurality of light-emitting material having different wavelengths is contained. For example, it may be possible to constitute the light-emitting layer by laminating a blue light-emitting layer, a green light-emitting layer and a red light-emitting layer, and then obtain the white color light emission by mixing the lights emitted from each light-emitting layer.

(Electron Transport Layer)

The electron transport layer is a layer which transports (injects) the electrons supplied from the second electrode 17 to the light-emitting layer. In addition, the electron transport layer acts as a barrier that blocks the inflow of the positive hole from the first electrode 15 side. The term of “electron transport layer” is sometimes used in a broad sense of including the electron-injecting layer and/or the positive hole-blocking layer.

Arbitrary material can be used as long as an electron transport material used in the electron transport layer adjacent to the second electrode 17 side of the light-emitting layer (the electron transport layer when the electron transport layer is in the single layer, and the electron transport layer positioned on a side closest to the light-emitting layer when the electron transport layer is disposed in a plural number) has a function that transmits (transports) the electron injected from the second electrode 17 to the light-emitting layer. For example, an arbitrary compound can be appropriately selected for use from among the well-known various compounds used in the conventional organic EL element, as the electron transport material.

More specifically, examples of the electron transport materials that can be used include metal complexes such as a fluorenone derivative, a carbazole derivative, an azacarbazole derivative, an oxazole derivative, a triazole derivative, a silole derivative, a pyridine derivative, a pyrimidine derivative, and an 8-quinolinone derivative. Examples of other electron transport materials that can also be used include a metal phthalocyanine or a metal-free phthalocyanine, or compounds obtained by substituting each of these terminals with an alkyl group, a sulfonic acid group or the like. In addition, in the present embodiment, for example, a dibenzofuran derivative can also be used as the electron transport material.

Furthermore, in the present embodiment, an electron transport layer having a high n-property (electron rich) may also be formed by doping of the electron transport layer with impurities as a guest material. Specific examples of the electron transport layers each having such a configuration are described in each of Japanese Patent Laid-Open Nos. 04-297076, 10-270172, 2000-196140, 2001-102175, as well as in J. Appl. Phys., 95, 5773 (2004). Specifically, an alkali metal salt of an organic substance can be used as the guest material (doping material).

When the alkali metal salt of an organic substance is used as the doping material, the kind of the organic substance is arbitrary, and examples of the usable organic substances include compounds including: a salt of formic acid, a salt of acetic acid, a salt of propionic acid, a salt of butylic acid, a salt of valelic acid, a salt of caproic acid, a salt of enanthic acid, a salt of caprylic acid, a salt of oxalic acid, a salt of malonic acid, a salt of succinic acid, a salt of benzoic acid, a salt of phthalic acid, a salt of isophthalic acid, a salt of terephthalic acid, a salt of salicylic acid, a salt of pyruvic acid, a salt of lactic acid, a salt of malic acid, a salt of adipic acid, a salt of mesylic acid, a salt of tosic acid, a salt of benzenesulfonic acid, and the like. Among them, preferable organic substance used is a salt of formic acid, a salt of acetic acid, a salt of propionic acid, a salt of butylic acid, a salt of valelic acid, a salt of caproic acid, a salt of enanthic acid, a salt of caprylic acid, a salt of oxalic acid, a salt of malonic acid, a salt of succinic acid, a salt of benzoic acid. Further preferable organic substance is an aliphatic carboxylic acid including: a salt of formic acid, a salt of acetic acid, a salt of propionic acid, a salt of butylic acid, a salt of valelic acid, a salt of caproic acid, a salt of enanthic acid, a salt of caprylic acid, a salt of oxalic acid, a salt of malonic acid, a salt of succinic acid, and a salt of benzoic acid, and when the aliphatic carboxylic acid is used, the number of carbon atoms is preferably 4 or less. Note that the most preferable organic substance includes a salt of acetic acid.

The kind of alkali metal which constitutes the alkali metal salt of the organic substance is arbitrary, and for example, Li, Na, K, or Cs can be used. Among these alkali metals, a preferred alkali metal is K or Cs, and more preferable alkali metal is Cs.

Accordingly, the alkali metal salt of the organic substance which can be used as the doping material of the electron transport layer is a compound obtained by combining the above-described organic substance and the above-described alkali metal. Specific examples of the doping materials that can be used include Li formate, K formate, Na formate, Cs formate, Li acetate, K acetate, Na acetate, Cs acetate, Li propionate, Na propionate, K propionate, Cs propionate, Li oxalate, Na oxalate, K oxalate, Cs oxalate, Li malonate, Na malonate, K malonate, Cs malonate, Li succinate, Na succinate, K succinate, Cs succinate, Li benzoate, Na benzoate, K benzoate, or Cs benzoate. Among them, the preferable doping material is Li acetate, K acetate, Na acetate or Cs acetate, and the most preferable doping material is Cs acetate. Note that a preferred content of the doping material is a value within the range of about 1.5 to 35% by mass with respect to the electron transport layer to which the doping material is added, more preferable content is a value within the range of about 3 to 25% by mass, and most preferable content is a value within the range of about 5 to 15% by mass.

(Electron-Injecting Layer)

In the organic EL element 10, the electron-injecting layer (electron buffer layer) may be disposed between the second electrode 17 and the light-emitting layer, or between the second electrode 17 and the electron transport layer. Similarly to the positive hole-injecting layer, the electron-injecting layer is disposed between the second electrode 17 and the organic compound layer (light-emitting layer or electron transport layer) in order to reduce a driving voltage and to enhance an emission luminance of the organic EL element 10.

Here, although the detailed explanation of the configuration of the electron-injecting layer is omitted, the specific configuration of the electron-injecting layer is described in detail, for example, in volume 2, chapter 2 “Electrode materials” (pp. 123-166) of “Organic EL Elements and Industrialization Front thereof” (Nov. 30, 1998, published by N.T.S. Co. Ltd.).

[Uses of Organic EL Element]

The organic EL elements 10 having the above-described embodiments are surface emitting elements as described above, and thus are usable for light-emitting sources of various types. Examples include, but are not limited to, a lighting device such as a home lighting device or a car lighting device, a backlight for a timepiece or a liquid crystal, a signboard for advertisement, a light source for a signal, a light source for an optical storage medium, a light source for an electrophotographic copier, a light source for an optical communication processor, a light source for an optical sensor, and the like. Particularly, the organic EL elements 10 can be effectively used as a backlight for a liquid crystal display device which is combined with a color filter and as a light source for lighting.

Furthermore, the organic EL element 10 may be used as a kind of lamp such as a lighting device or a light source for exposure, or may be used as a projection device of an image projecting type, or as a display device of a type that a still image or moving image is directly and visually recognized. In the case, a light-emitting surface area may be enlarged by so-called tiling in which light-emitting panels with the organic EL element 10 are combined flatly along with the recent size enlargement of lighting devices and displays.

A driving system in the case of the use as a display device for reproducing a moving image is either a simple matrix (passive matrix) system or active matrix system. Furthermore, it is possible to produce a color or full color display device by using two or more kinds of the organic EL element 10 having a different color emission.

Hereinafter, a lighting device will be explained as one example of the uses, and next, a lighting device having an emission area enlarged by tiling will be explained.

(Lighting Device)

The organic EL element 10 in the above embodiments can be applied to a lighting device.

The lighting device using the above-described organic EL element 10 may be designed so as to impart a resonator structure to the each organic EL element of the above-described construction. The objects to be used of the organic EL element 10 having the resonator structure include a light source for an optical storage medium, a light source for an electrophotographic copier, a light source for an optical communication processor, a light source for an optical sensor, and the like, but are not limited thereto. Alternately, the organic EL element may be used for the above-described use by achieving laser oscillation.

Note that the material used for the organic EL element 10 can be applied to an organic EL element which emits a substantial white light (also referred to as white organic EL element). For example, a plurality of emission colors is emitted at the same time from a plurality of light-emitting materials to prepare a white color emission by color mixing. Examples of the combination of a plurality of emission colors may include a combination containing three maximum emission wavelengths of three primary colors of red, green and blue, or a combination containing two maximum emission wavelengths which are in complementary color relation such as blue and yellow, bluish green and orange, or the like.

Moreover, the combinations of light-emitting materials for obtaining a plurality of emission colors are either a combination of a plurality of light-emitting materials which emits a plurality of phosphorescence or fluorescence, or a combination of a light-emitting material which emit a plurality of phosphorescence or fluorescence and a dye material which emits excitation light from a light-emitting material, and may be a combination of a plurality of luminous dopants in the white color organic EL element.

The white color organic EL element as described above has a configuration different from the configuration in which a white color emission is obtained by arranging organic EL elements each of which emits individual color light in parallel array, and emits white color light from the organic EL element itself. Therefore, it is not necessary to use a mask in order to form most of all layers constituting the element. Accordingly, for example, the electrode layer can be formed all over one surface by a vapor deposition method, a casting method, a spin coating method, an ink-jet method, a printing method, and the like, which enhances productivity.

Furthermore, the materials to be used for the light-emitting layers of the white color organic EL element are not particularly limited. For example, in a case of a backlight in a liquid crystal display element, an arbitrary material is selected from among the above-described metal complexes or well-known light-emitting materials, for the combination, so as to satisfy a wavelength range corresponding to a CF (color filter) property to thereby perform whitening.

It is possible to produce a lighting device which emits substantial white light by using the white color organic EL element explained above.

### EXAMPLE

Hereinafter, although the present invention will be specifically explained by referring Examples, the present invention is not limited thereto.

<Production of Organic EL Element>

The organic EL elements of Samples 101 to 109 were fabricated according to the following manner. Hereinafter, the configurations and production procedures of the organic EL elements of the Samples 101 to 109 will be shown. Besides, the word “%” used in the following Examples means “% by mass”, otherwise noted.

[Production of Organic EL Element of Sample 101]

(Substrate)

A biaxially oriented polyethylene naphthalate film (PEN film, thickness: 100 μm, width: 350 mm, manufactured by Teijin DuPont Films Co., Ltd., trade name “Teonex Q65FA”) was prepared as the substrate.

(Primer Layer)

OPSTAR Z7501 (UV curable organic/inorganic hybrid hard coating material, manufactured by JSR Co., Ltd.) was coated, by a wire bar, on an easily-adhering surface of the substrate so that the layer thickness after drying was 4 μm by a wire bar. After drying under the drying conditions of 80° C. for 3 minutes, a primer layer was formed by curing under the curing condition of 1.0 J/cm² by using a high-pressure mercury lamp, in an air atmosphere.

At this time, a maximum cross-sectional height Ra(p) which represents surface roughness was 5 nm.

Note that the surface roughness (arithmetic mean roughness Ra) is measured by using an atomic force microscope (manufactured by Digital Instruments Co., Ltd.), and calculated from an uneven cross-sectional curve continuously measured with a detection device having a sensing pin with a minimum tip radius. The measurement was performed three times within a zone of 30 μm in the measuring direction with the sensing pin with a minimum tiny tip radius, and the surface roughness was calculated from average roughness relating to amplitude of fine unevenness.

(First Gas Barrier Layer)

The substrate obtained by forming the primer layer was mounted on a CVD apparatus, and then a first gas barrier layer was produced at a thickness of 300 nm on the substrate under the following deposition conditions (plasma CVD conditions) so as to have each element profile shown in FIG. 5.

Deposition Conditions

Supply amount of hexamethyldisiloxane (HMDSO, (CH₃)₆SiO)) (raw material gas): 50 sccm (Standard Cubic Centimeter per Minute)

Supply amount of oxygen gas (O₂): 500 sccm

Degree of vacuum in vacuum chamber: 3 Pa

Applied power from plasma generation power source: 0.8 kW

Frequency of plasma generation power source: 80 kHz

Conveyance speed of film: 0.5 to 1.66 m/min

(Second Gas Barrier Layer)

A 10% by mass solution of perhydropolysilazane (PHPS) (AQUAMICA NN120-10, non-catalyst type, manufactured by AZ made Electronic Materials Co., Ltd.) in dibutyl ether was used as a coating solution.

Then, the above coating solution was coated on the first gas barrier layer by using a wire bar so that an (average) layer thickness after drying was 300 nm, and was dried after treatment for 1 minute under an atmosphere of a temperature of 85° C., and a humidity of 55% RH. Furthermore, a polysilazane layer was formed by subjecting the resultant substance to dehydration treatment after holding the substance for 10 minutes under an atmosphere of a temperature of 25° C., a humidity of 10% RH (dew point −8° C.)

Next, the polysilazane layer thus produced was subjected to the silica conversion treatment under atmospheric pressure by using the following ultraviolet ray irradiation apparatus. Additionally, in the ultraviolet ray irradiation apparatus, the substrate obtained by forming the polysilazane layer and fixed to the operation stage was subjected to modification treatment under the following conditions to thereby form a second gas barrier layer.

### Ultraviolet Ray Irradiation Apparatus

Apparatus: Excimer irradiation apparatus MODEL MECL-M-1-200 manufactured by M D COM Co., Ltd.

Irradiating wavelength: 172 nm

Lamp filler gas: Xe

### Modification Treatment Conditions

Excimer lamp light intensity: 130 mW/cm² (172 nm)

Distance between sample and light source: 1 mm

Stage heating temperature: 70° C.

Oxygen concentration in irradiation apparatus: 1.0%

Excimer lamp irradiation time: 5 seconds

The above substrate obtained by forming layers up to the second gas barrier layer had a water vapor permeability of less than 1×10⁻⁴ g/(m²·24 h), and exhibited very good water vapor barrier properties.

Note that, in this Example, the water vapor permeability was a value measured by the method in accordance with JIS K 7129-1992 at a temperature of 25±0.5° C., a relative humidity of 90±2% RH.

(First Electrode)

Next, the above substrate obtained by forming the layers up to the gas barrier layer was cut to a size of 5 cm×5 cm, and was fixed to a substrate holder of the commercially available vacuum deposition apparatus. Furthermore, the following Compound (1-6) was contained in a resistive heating boat made of tantalum. Then, these substrate holder and resistive heating boat were attached to a first vacuum tank of the vacuum deposition apparatus.

In addition, silver (Ag) was contained in a resistive heating boat of tungsten, and was attached to a second vacuum tank of the vacuum deposition apparatus.

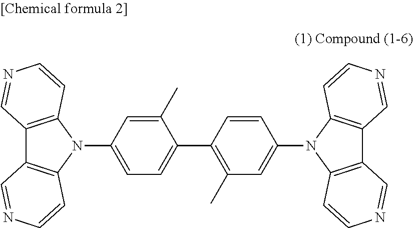 [SMILES: Cc1cc(-n2c3ccncc3c3cnccc32)ccc1-c1ccc(-n2c3ccncc3c3cnccc32)cc1C]

Next, after a pressure of the first vacuum tank was reduced to 4×10⁻⁴ Pa, the heating boat containing the Compound (1-6) was heated by applying an electric current, and then there was formed, on the second gas barrier layer, the base layer made of the Compound (1-6) for the first electrode at a deposition rate of 0.1 to 0.2 nm/sec. A thickness of the base layer was 50 nm.

Subsequently, the substrate obtained by forming the layers up to the base layer was transferred to the second vacuum tank under vacuum. After a pressure of the second vacuum tank was reduced to 4×10⁻⁴ Pa, the heating boat containing silver was heated by applying an electric current, and then there was formed, on the base layer, an electrode layer made of silver having a thickness of 8 nm at a deposition rate within the range of 0.1 to 0.2 nm/sec to thereby give an electrode layer of the first electrode (anode).

(Organic Function Layer)

The constituent material for each layer of the organic function layer was filled in crucibles for vapor deposition in the vacuum deposition apparatus, in an optimum amount for producing the respective organic EL elements. The constituent material for each layer of the organic function layer was filled in the crucibles for vapor deposition made of resistive heating material such as molybdenum or tungsten.

There were used, as the constituent material for each layer of the organic function layer, the compound α-NPD, the compound BD-1, the compound GD-1, the compound RD-1, the compound H-1, the compound H-2 and the compound E-1, described below.

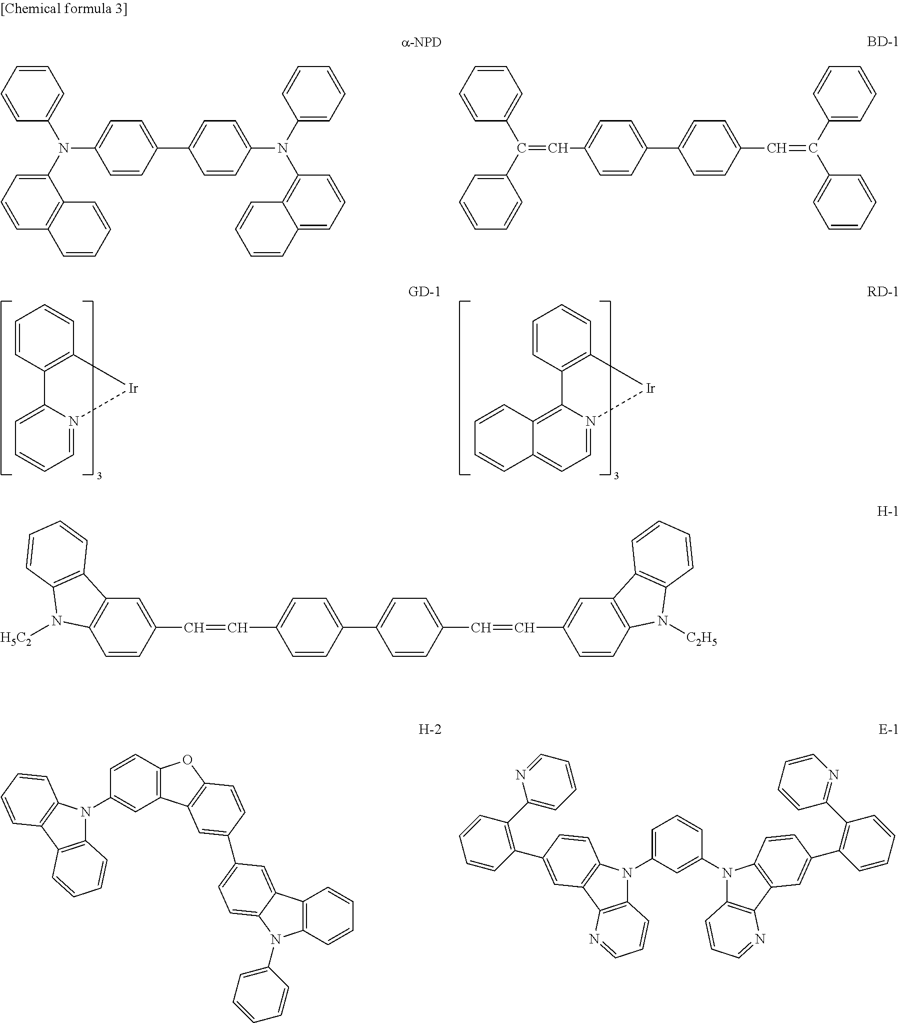

First, the pressure of the vacuum chamber of the vacuum deposition apparatus was reduced to a degree of vacuum of 1×10⁻⁴ Pa, the crucible for vapor deposition filled with the compound α-NPD was heated by passing a current, and the α-NPD was deposited on the first electrode at a deposition rate of 0.1 nm/sec, with the result that a positive hole injection transport layer having a thickness of 40 nm was formed.

Similarly, the compound BD-1 and the compound H-1 were co-deposited at a deposition rate of 0.1 nm/sec so that the concentration of the compound BD-1 was 5%, and thus a fluorescent blue light-emitting layer having a thickness of 15 nm was formed.

Next, the compound GD-1, the compound RD-1 and the compound H-2 were co-deposited at a deposition rate of 0.1 nm/sec so that the concentration of the compound GD-1 was 17% and the concentration of the compound RD-1 was 0.8%, and thus a phosphorescent yellow light-emitting layer having a thickness of 15 nm was formed.

After that, the compound E-1 was deposited at a deposition rate of 0.1 nm/sec, and thus an electron transport layer having a thickness of 30 nm was formed.

(Second Electrode)

Next, a lithium fluoride (LiF) layer was formed at a thickness of 1.5 nm on the organic function layer, and an aluminum layer having a thickness of 110 nm was deposited and a second electrode (cathode) was formed by vapor deposition of an aluminum layer having a thickness of 110 nm. The second electrode was formed in the form in which the terminal part was led out from the edge of the substrate, in a state of being electrically insulated from the first electrode by the organic function layer from the positive hole-injecting layer to the electron-injecting layer.

Note that a vapor deposition mask was used for forming the first electrode, the organic function layer and the second electrode. In addition, a region of 4.5 cm×4.5 cm positioned at the center of the 5 cm×5 cm substrate was set as a light-emitting region, and a non-light-emitting region having a width of 0.25 cm was provided around the whole of the light-emitting region.

(Sealing)

First, an adhesive composition having a solid content of about 25% by mass was prepared by dissolving, in toluene, 100 parts by mass of “Oppanol B50 (manufactured by BASF made, Mw: 340000)” as a polyisobutylene-based resin, 30 parts by mass of “Nisseki Polybutene Grade HV-1900 (manufactured by JX Nippon Oil & Energy Corporation, Mw: 1900) as a polybutene resin, 0.5 part by mass of “TINUVIN 765 (Ciba Japan KK) as a hindered amine-based photostabilizer, 0.5 part by mass of “IRGANOX 1010 (manufactured by Chiba Japan KK, the β-positions of the hindered phenol group being both tertiary butyl groups) as a hindered phenol-based antioxidant, and 50 parts by mass of “EASTOTAC H-100L Resin (manufactured by Eastman Chemical Co., Ltd.)” as a cyclic olefin-based polymer.

Next, as a sealing member, the solution of the adhesive composition prepared above was applied to an aluminum (Al) side of an aluminum-deposited polyethylene terephthalate film “Alpet 12/34 (manufactured by Asia-Alumi Co., Ltd.) so that a dry thickness of the adhesive layer was 20 μm, and dried at 120° C. for 2 minutes to form an adhesive layer. Next, a peeling-off treated surface of a peeling-off treated polyethylene terephthalate film having a thickness of 38 μm as a peeling-off sheet adhered to the pressure adhesive layer surface to form a pressure sensitive adhesive sheet for sealing.

Next, after the peeling-off sheet of the above prepared pressure sensitive adhesive sheet for sealing was removed under a nitrogen atmosphere, the pressure sensitive adhesive sheet for sealing was dried on a hot plate heated at 120° C. for 10 minutes, and, after confirming that the sheet was cooled to room temperature (25° C.), the second electrode was completely laminated with the pressure sensitive adhesive sheet for sealing, and then heated at 90° C. for 10 minutes to seal the organic EL element.

(Externally Light Taking-Out Layer)

Next, the organic EL element of Sample 101 was produced by sticking a Micro lens array sheet (manufactured by MNtech Co., Ltd.), as an externally light taking-out layer, to a surface of the substrate opposite to the surface where respective layers were formed. In the organic EL element of the Sample 101, each color of lights emitted from the light-emitting layer can be taken out from the first electrode side, namely, from the substrate side.

[Production of Organic EL Element of Sample 102]

A smooth layer was formed according to the following procedures on the substrate obtained by forming the layers up to the second gas barrier layer in a similar way to that in the above Sample 101. Then, the first electrode, the organic function layer and the second electrode were produced on thus produced smooth layer in a similar way to that in Sample 101, and the organic EL element of Sample 102 was formed by formation of the externally light taking-out layer after sealing with the adhesive sheet for sealing.

(Smooth Layer)

First, an organic solvent was prepared as a coating solution for forming the smooth layer so that 1-butanol/hexylene glycol/propylene glycol propyl ether were mixed in a ratio of 1/1/1, and formulation design was performed in a ratio of an amount of 10 ml so that a solid content of a thermosetting oligomer having a high refractive index (Titanium oxide film forming agent PC-200, manufactured by Matsumoto Fine Chemical Co., Ltd.) was 12% by mass in the organic solvent.

Specifically, the above thermosetting resin having a high refractive index was mixed with the solvent, and after stirring at 500 rpm for 1 minute, the resultant mixture was filtered by a hydrophobic PVDF 0.2 μm filter (manufactured by Whatman Co., Ltd.) to give a desired coating solution.

Next, the above coating solution was coated using the inkjet coating method in a desired pattern to form the coating film. Then, after simple drying (80° C. for 2 minutes), and furthermore, drying treatment was performed for 5 minutes under the output condition of less than 80° C. of the substrate temperature, by using wavelength-controllable IR.

In addition, a smooth layer was formed by holding the dried coating film over one day in an atmosphere of normal temperature and normal humidity and by accelerating the curing reaction.

The drying treatment by the wavelength-controllable IR was performed by attaching two quartz glass plates which absorb an infrared ray having a wavelength of 3.5 μm or more to a radiant heat transfer machine with a wavelength-controllable infeed ray heater (IR radiation machine, Ultimate heater/carbon, manufactured by MEI MEI INDUSTRIES INC.), and by allowing a cooling air to flow between the glass plates. At this time, the cooling air was allowed to flow at 200 L/min, and a temperature of the quartz glass on the tube surface was suppressed to be less than 120° C. The temperature of the substrate was measured by arranging the k thermocouples on the upper and lower surfaces of the substrate and above the substrate by 5 mm, and by connecting to a NR2000 (KEYENCE CORPORATION INC.).

[Production of Organic EL Element of Sample 103]

Through the use of a glass substrate as the substrate, the layers up to the second gas barrier layer were formed on this glass plate in a similar way to that in the above Sample 101. Then, the smooth layer was produced on the second gas barrier layer according to the following way. Furthermore, the first electrode, the organic function layer and the second electrode were produced on thus produced smooth layer in a similar way to that in the above Sample 101, and the organic EL element of Sample 103 was formed by formation of the externally light taking-out layer after sealing with the adhesive sheet for sealing.

(Smooth Layer)

In a similar way to that in the above Sample 102, the preparation of a coating solution, production of a coating film, and the drying treatment were performed, and after that, the resultant layer was heated and dried on the hot plate at 400° C. for one hour in an environment of normal temperature and normal humidity, and a smooth layer was formed by acceleration of a curing reaction of the coating film after drying.

[Production of Organic EL Element of Sample 104]

A smooth layer was formed according to the following procedures on the substrate obtained by forming the layers up to the second gas barrier layer in a similar way to that in the above Sample 101. Then, the first electrode, the organic function layer and the second electrode were produced on thus produced smooth layer in a similar way to that in the above Sample 101, and the organic EL element of Sample 104 was formed by formation of the externally light taking-out layer after sealing with the adhesive sheet for sealing.

(Smooth Layer)

In a similar way to that in the above Sample 102, the preparation of a coating solution, production of a coating film, and the drying treatment were performed, and after that, the resultant layer was subjected to modification treatment by the excimer light under the following conditions and a smooth layer was formed by acceleration of a curing reaction of the coating film after drying.

### Modification Treatment Apparatus

Apparatus: Excimer irradiation apparatus MODEL MECL-M-1-200 manufactured by M D COM Co., Ltd.

Irradiating wavelength: 172 nm

Lamp filler gas: Xe

### Modification Treatment Conditions

Excimer lamp light intensity: 130 mW/cm² (172 nm)

Distance between sample and light source: 1 mm

Stage heating temperature: 70° C.

Oxygen concentration in irradiation apparatus: Air

Excimer light energy: 8 J/cm² (172 nm)

[Production of Organic EL Element of Sample 105]

A smooth layer was formed according to the following procedures on the substrate obtained by forming the layers up to the second gas barrier layer in a similar way to that in the above Sample 101. Then, the first electrode, the organic function layer and the second electrode were produced on thus produced smooth layer in a similar way to that in Sample 101, and the organic EL element of Sample 105 was formed by formation of the externally light taking-out layer after sealing with the adhesive sheet for sealing.

(Smooth Layer)

First, a coating solution was prepared by diluting tetraxis(2-ethylhexanoic acid) titanium (IV) with methanol twice. A coating film was formed by spin-coating the coating solution at 4000 rpm. Then, the coating film was subjected to drying treatment by simple drying (80° C. for 2 minutes) and then by performing drying for 5 minutes under the output condition of less than 80° C. of the substrate temperature by using wavelength-controllable IR.

Next, the dried coating film was irradiated with ArF excimer laser (193 nm) light in an atmosphere at 50 Hz, 10 mJ/cm² for 60 seconds and further at 10 Hz, 50 mJ/cm² for 15 minutes, with the result that the smooth layer made of the anatase-type crystalline film was formed.

[Production of Organic EL Element of Sample 106]

A light-scattering layer was formed according to the following procedures on the substrate obtained by forming the layers up to the second gas barrier layer in a similar way to that in the above Sample 101. Then, in a similar way to that in the above Sample 102, the smooth layer was formed on the thus produced light-scattering layer. Furthermore, the first electrode, the organic function layer and the second electrode were produced on thus produced smooth layer in a similar way to that in the above Sample 101, and the organic EL element of Sample 106 was formed by formation of the externally light taking-out layer after sealing with the adhesive sheet for sealing.

(Light-Scattering Layer)

First, TiO₂ particles having a refractive index of 2.4 and an average particle size of 0.25 μm (JR600A manufactured by TEICA CORPORATION) as the light-scattering particle and a resin solution ED230AL (organic inorganic hybrid resin manufactured by APM Corporation) were mixed in a solid content ratio of 40% by volume/60% by volume, and then the resultant mixture was prepared so that a solid content in propylene glycol monomethyl ether (PGME) was 15% by mass.

Formulation design was performed in a ratio of an amount of 10 ml by adding 0.4% by mass of Disperbyk-2096 (manufactured by Byk Chemi Japan Co., Ltd.) as an additive relative to the above solid component (effective mass component).

Specifically, the above solvent and the additives were mixed in a mass ratio of 10% relative to the TiO₂ particles, and the resultant mixture was dispersed while being cooled at normal temperature (25° C.) for 10 minutes by using an ultrasonic dispersing machine (UH-50 manufactured by SMT Co., Ltd.) under the standard conditions of microchip step (MS-3 3 mmφ) to produce a TiO₂ dispersion.

Next, the resin solution was added little by little to the TiO₂ dispersion while the dispersion being stirring at 100 rpm, and after the completion of the addition, the stirring speed of the resultant mixture was raised to 500 rpm and then the mixture was stirred for 10 minutes and filtered by a hydrophobic PVDF 0.45 μm filter (manufactured by Whatman Co., Ltd.) to thereby give a desired coating solution for forming of the light-scattering layer.

Subsequently, the above coating solution was coated onto the second gas barrier layer according to the inkjet coating method to thereby form the coating film, and was then subjected to simple drying (80° C. for 2 minutes). Furthermore, the resultant coating film was subjected to drying treatment for 5 minutes under the output condition of less than 80° C. of the substrate temperature by using wavelength-controllable IR.

Then, the modification treatment by the excimer light was performed on the produced coating film under the following conditions, and then a light-scattering layer having a thickness of 0.3 μm was formed by accelerating the curing reaction of the dried coating film.

### Modification Treatment Apparatus

Next, the curing reaction was accelerated under the following modification treatment conditions.

Apparatus: Excimer irradiation apparatus MODEL MECL-M-1-200 manufactured by M D COM Co., Ltd.

Irradiation wavelength: 172 nm

Lamp filler gas: Xe

### Modification Treatment Conditions

Excimer lamp light intensity: 130 mW/cm² (172 nm)

Distance between sample and light source: 1 mm

Stage heating temperature: 70° C.

Oxygen concentration in irradiation apparatus: Air

Excimer light energy: 8 J/cm² (172 nm)

[Production of Organic EL Element of Sample 107]

A light-scattering layer was formed in a similar way to that in the above Sample 106 on the substrate obtained by forming the layers up to the second gas barrier layer in a similar way to that in the above Sample 103. Then, in a similar way to that in the above Sample 103, the smooth layer was formed on the thus produced light-scattering layer. Furthermore, the first electrode, the organic function layer and the second electrode were produced on thus produced smooth layer in a similar way to that in the above Sample 101, and the organic EL element of Sample 107 was formed by formation of the externally light taking-out layer after sealing with the adhesive sheet for sealing.

[Production of Organic EL Element of Sample 108]

A light-scattering layer was formed in a similar way to that in the above Sample 106 on the substrate obtained by forming the layers up to the second gas barrier layer in a similar way to that in the above Sample 101. Then, in a similar way to that in the above Sample 104, the smooth layer was formed on the thus produced light-scattering layer. Furthermore, the first electrode, the organic function layer and the second electrode were produced on thus produced smooth layer in a similar way to that in the above Sample 101, and the organic EL element of Sample 108 was formed by formation of the externally light taking-out layer after sealing with the adhesive sheet for sealing.

[Production of Organic EL Element of Sample 109]

A light-scattering layer was formed in a similar way to that in the above Sample 106 on the substrate obtained by forming the layers up to the second gas barrier layer in a similar way to that in the above Sample 101. Then, in a similar way to that in the above Sample 105, the smooth layer was formed on the thus produced light-scattering layer. Furthermore, the first electrode, the organic function layer and the second electrode were produced on thus produced smooth layer in a similar way to that in the above Sample 101, and the organic EL element of Sample 109 was formed by formation of the externally light taking-out layer after sealing with the adhesive sheet for sealing.

[Evaluation of Organic EL Element of Each Sample]

The produced organic EL elements of the Samples 101 to 109 were subjected to the following property evaluation. The results of each sample are shown in Table 1.

(Raman Spectroscopic Absorption)

The condition of the smooth layer of each sample of the organic EL element was measured by using a microlazer Raman spectroscope: Almega XR manufactured by Thermo Fisher Scientific Co., Ltd. The measurement laser wavelength was 532 nm.

Note that the measurement was performed at the time when layers up to the smooth layer were formed in the above-described formation processes of the organic EL element.

(Spectroscopic Ellipsometer: nk Measurement)

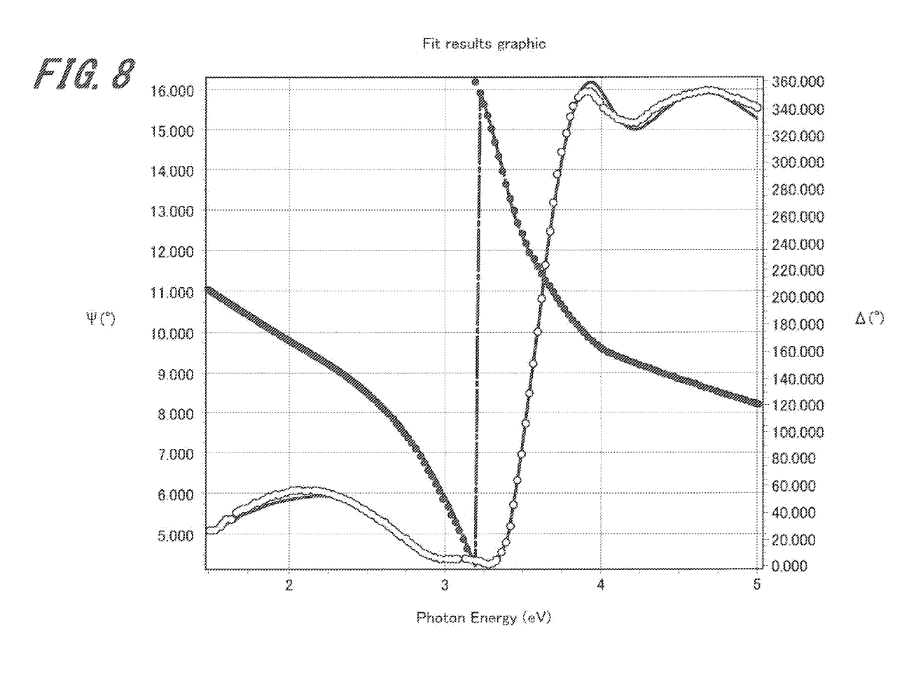

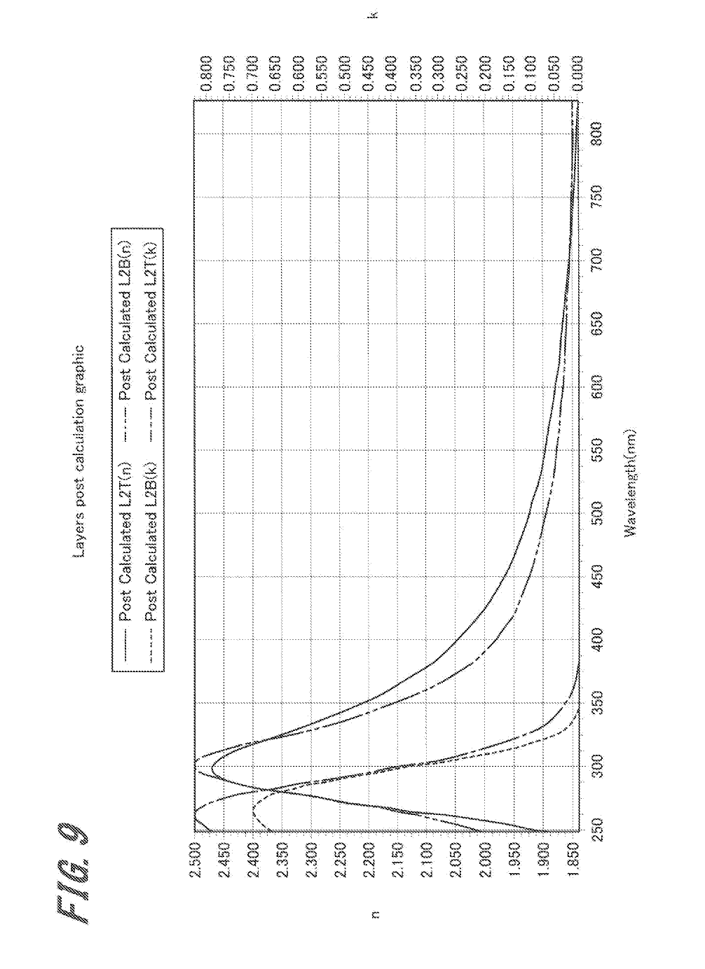

The nk value of the smooth layer of each Sample of the organic EL element was measured by using a spectroscopic ellipsometer UVSEL/FUV-FGMS manufactured by HORIBA JYOBIN-YVON Co., Ltd., and by performing fitting as shown in FIG. 8 and FIG. 9. It is found that the highly transparent amorphous layer having a high refractive index is formed from the results of the Raman spectrum, the fitting accuracy of the ellipso, and the nk value obtained therefrom.

Note that the measurement was performed at the time when the smooth layer was formed in the production processes of the above-described organic EL element.

(Flexibility Adequacy)

There was repeated a procedure in which the organic EL element of each sample produced was wound around a roller having 25 mmcp by 100 rotations in a state where the base material was allowed to face a roller side, and subsequently, the resultant wound element was all returned to a flat state. The presence or absence of crack was observed by an optical microscope after repeating the aptitude test 100 times. The flexibility adequacy was evaluated according to the following evaluation standard.

◯: No damage
Δ: Slight crack at the edge
x: Many cracks

(Element Reflectivity)

The element reflectivity was evaluated by introducing light to the organic EL element from the light taking-out surface, and measuring the light reflected on the second electrode, or the like, and emitted from the organic EL element.

x: Standard level
Δ: Reflectivity being higher than standard by 5% or more
◯: Reflectivity being higher than standard by 10% or more

(Light Emission Efficiency)

With respect to the organic EL element of the fabricated each sample, the light emission test was conducted by lighting at room temperature (25° C.) under the constant current density condition of 2.5 mA/cm², measuring the light emission luminance with a spectroscopic radiant luminance meter CS-2000 (manufactured by Konica Minolta, Inc.), and then calculating the light emission efficiency (externally taking-out efficiency) at the current value.

Note that the light emission efficiency is expressed as a relative value that the light emission efficiency of the organic EL element of Sample 101 is 100.

### (Dark Spot (DS))

Under the same constant current density conditions as those in the above measurement of the light emission efficiency, the states of generation of the dark spot (DS) at the initial stage of the light emission, and after 200 hours from charging a thermostatic tank at 85° C. (dry) with the organic EL element of each sample were observed with naked eyes, and then evaluated according to the following standard.

◯: Absence of DS having 150 μmφ or more
◯Δ: 1 to 3 of DS having 150 μmφ or more
Δ: 4 to 20 of DS having 150 μmφ or more
Δx: many DS having 150 μmφ or more
x: Not emitted

(Weather Resistance)

The organic EL element of the fabricated each sample was subjected to the irradiation conducted by using “Xenon Weather Meter XL75” (manufactured by Suga Test Instruments Co., Ltd.) under the irradiation condition of 70,000 lux xenon lamp for one month. After the irradiation, the organic EL element of each sample was driven, the state of the element after the weather resistance test was observed with naked eyes.

◯: Basically driving without trouble
◯Δ: Generation of degradation slightly (less than 5%)
Δ: Generation of degradation partially (less than 20%)
Δx: Generation of degradation in a large area (less than 50%)
x: Not driven

TABLE 1

| | | | | Raman spectroscopic | | | | | | | |
|---|---|---|---|---|---|---|---|---|---|---|---|
| | Light- | | | absorption | | | | | Light | | |
| | scattering | Smooth | Curing | Organic | | Spectroscopic | Flexibility | Element | emission | | Weather |
| Sample | layer | layer | treatment | substance | TiO₂ | ellipsometer n | adequacy | reflectivity | efficiency | DS | resistance |
| 101 | Absence | Absence | — | — | — | — | ◯ | X | 100 | ◯ | ◯ |
| 102 | Absence | Presence | Drying | Remaining | Oligomer | 1.6 | ◯ | X | — | X | X |
| 103 | Absence | Presence | Thermosetting | Absence | Crystal | 2.1 | X | ◯ | 120 | ◯ | X |
| 104 | Absence | Presence | Excimer treatment | Absence | Amorphous | 1.85 | ◯ | ◯ | 115 | ◯ | ◯ |
| 105 | Absence | Presence | Laser treatment | Absence | Crystal | 1.9 | X | ◯ | 118 | ◯ | X |
| 106 | Presence | Presence | Drying | Remaining | Oligomer | 1.6 | ◯ | X | — | X | X |
| 107 | Presence | Presence | Thermosetting | Absence | Crystal | 2.1 | X | ◯ | 160 | ◯ | X |
| 108 | Presence | Presence | Excimer treatment | Absence | Amorphous | 1.85 | ◯ | ◯ | 160 | ◯ | ◯ |
| 109 | Presence | Presence | Laser treatment | Absence | Crystal | 1.9 | X | ◯ | 155 | ◯ | X |

With respect to the organic EL elements of the Sample 102 and the Sample 106 where the smooth layer was formed by drying the smooth layer over one day under normal temperature and normal humidity, since the TiO₂ was not crystallized well to be still as the Ti oligomer, the elements still had a low refractive index of 1.6. Further, since the crystallization reaction of the TiO₂ did not have been completed yet, the organic substance remained in the layer yielded, and a large amount of outgas which was released from the layer and gave adverse effect to the organic EL element, many dark spots were generated, and the organic EL element was not emitted.

With respect to the organic EL elements of the Sample 103, the Sample 105, the Sample 107 and the Sample 109 where the TiO₂ crystal layer were formed by the heat curing or the laser irradiation, the refractive index became higher due to the crystallization of the TiO₂. However, with respect to the Sample 103 and the Sample 107 where the crystal layer was formed on the glass substrate by the heat curing, the smooth layer was broken at the time when the glass substrate was broken. Also with respect to the Sample 105 and the Sample 109 where the crystal layer was formed on the PEN film by the laser irradiation, the smooth layer was broken at the flexibility adequacy test.

As described above, with respect to the samples of the organic EL elements having the crystallized smooth layer, the bad results were found in the flexibility adequacy due to hardness and brittle property of the TiO₂ crystallized layer. Further, due to low flexibility of the smooth layer, the bad results such as peeling off were yielded in the weather resistance.

In contrast, with respect to the organic EL elements of Sample 104 and Sample 108 where the amorphous smooth layer was formed by the excimer treatment, even if the layer was amorphous, the refractive index of the smooth layer was increased. In addition, since the organic substance was not remained and the result of the generation of dark spot was good, it has been found that the generation of the outgas as in the Sample 102 and the Sample 106 was not found.

Furthermore, from the results of the flexibility adequacy, the smooth layer of the organic EL element has enough flexibility.

In addition, in the light emission efficiency and the element reflectivity, the organic EL elements of the Sample 103, the Sample 105, the Sample 107 and the Sample 109 having the TiO₂ crystal layers as the smooth layers have the same results.

Accordingly, it is possible to impart the flexibility adequacy to the organic EL element, by using the smooth layer having the amorphous structure through the excimer treatment, without lowering the properties of the organic EL element.

Note that the present invention is not limited to the constructions explained in the above embodiments, and other various modification and change can be made within the scope of the present invention.

### REFERENCE SIGNS LIST

10 Organic EL element
11 Substrate
12 Gas barrier layer
13 Light-scattering layer
14 Smooth layer
15 First electrode
16 Organic function layer
17 Second electrode
20 delivery roller
21, 22, 23, 24 conveyance roller
25 winding roller
31, 32 Film-deposition roller
41 Gas inlet
51 Power source for plasma generation
61, 62 Magnetic-field generator

1. An organic electroluminescent element, comprising:
a substrate;
a gas barrier layer that is arranged on the substrate;
a smooth layer that is mainly composed of an oxide or nitride of Ti or Zr having an amorphous structure;
a first electrode;
a second electrode; and
an organic function layer that is sandwiched between the first electrode and the second electrode.

2. The organic electroluminescent element according to claim 1, wherein there is provided a light-scattering layer which contains a light-scattering particle and a resin, and which has a refractive index of 1.6 or more and 3.0 or less, between the gas barrier layer and the smooth layer.

3. The organic electroluminescent element according to claim 1, wherein at least any one of the first electrode and the second electrode is mainly composed of Ag.

4. The organic electroluminescent element according to claim 1, wherein the smooth layer is formed of a compound which has at least any one selected from an alkoxide structure and a chelate structure.

5. The organic electroluminescent element according to claim 4, wherein the compound which has at least any one selected from an alkoxide structure and a chelate structure is mainly composed of an oligomer having a polymerization degree of 3 or more and 30 or less.

6. The organic electroluminescent element according to claim 1, wherein the smooth layer is modified into an amorphous oxide or an amorphous nitride by an electron beam or excimer light having a wavelength of 150 nm or more and 250 nm or less.
